(19) United States
(12) Patent Application Publication
(10) Pub. No.: US 2021/0260242 A1
(43) Pub. Date: Aug. 26, 2021

(54) BIOACTIVE IMPLANTABLE DEVICES, AND COMPOSITE BIOMATERIALS AND METHODS FOR MAKING THE SAME

(71) Applicant: PROSIDYAN, INC., New Providence, NJ (US)

(72) Inventors: Charanpreet S. Bagga, Basking Ridge, NJ (US); Robert T. Warren, Bridgewater, NJ (US); Hyun W. Bae, Los Angeles, CA (US)

(21) Appl. No.: 17/182,920

(22) Filed: Feb. 23, 2021

Related U.S. Application Data
(60) Provisional application No. 62/980,805, filed on Feb. 24, 2020.

(51) Int. Cl. A61L 27/10; A61L 27/44; A61L 27/58; A61L 27/56; A61L 27/54

(52) U.S. Cl. CPC A61L 27/10; A61L 27/446; A61L 27/54; A61L 27/56; A61L 27/58

(57) ABSTRACT

Implantable medical devices, composite bioactive polymeric biomaterials for forming such devices, and methods for manufacturing these biomaterials and devices are provided. The implantable medical devices are engineered, at least in part, from a composite material comprising a polymer component and a bioactive component incorporated therein to provide bioactivity to the polymeric component for the improved treatment of bone or other purposes. The implantable device may comprise a main body formed of a polymeric framework, and a bioactive glass additive incorporated into the rigid polymeric framework. The implantable device may also comprise a main body and a bioactive component that includes a polyarylretherketone (PAEK) polymer component and a bioactive additive component. The bioactive additive component is incorporated substantially throughout the polymer component to further enhance cellular activity to promote bone fusion and/or regeneration.

### CROSS-REFERENCE TO RELATED APPLICATIONS

The present application claims the benefit of U.S. Provisional Application No. 62/980,805, filed Feb. 24, 2020, the complete disclosure of which is incorporated herein by reference in its entirety for all purposes as if copied and pasted herein.

The present application is also related to commonly-assigned, co-pending U.S. application Ser. No. 16/294,138, filed Mar. 6, 2019, Ser. No. 16/695,997, filed Nov. 27, 2019 and Ser. No. 16/151,774, filed Oct. 4, 2018 and commonly-assigned U.S. Pat. Nos. 9,381,274, 8,889,178, 8,883,195, 9,339,392 and 8,567,162, each of which is incorporated herein by reference in its entirety for all purposes as if copied and pasted herein.

### TECHNICAL FIELD

The present disclosure relates to implantable medical devices, biomaterials for forming such devices, and methods for manufacturing such biomaterials and devices. More particularly, the disclosure relates to implantable medical devices formed from a composite biomaterial that includes a polymer component and a bioactive component incorporated therein to provide bioactivity to the polymer component for the improved treatment of bone or other purposes.

### BACKGROUND

Biomaterials such as biocompatible metals and polymers have been used as implants in the field of spine, orthopedics and dentistry for over a century, including for use in trauma, fracture repair, reconstructive surgery, repairing or replacing damaged bone and alveolar ridge reconstruction. Although metal implants have been the predominant implants of choice for load-bearing applications, additional ceramics and nonresorbable polymeric materials have been employed within the last twenty-five years due to their biocompatibility and physical properties.

For example, polyaryletherketone (PAEK) polymers are often used to make medical implants. These PAEK polymers, which include polyetheretherketone (PEEK) and polyetherketoneketone (PEKK), can be molded into preselected shapes that possess desirable load-bearing properties. PEEK is a thermoplastic with excellent mechanical properties, including a Young's modulus of about 3.6 GPa and a tensile strength of about 100 MPa. PEEK is semi-crystalline, melts at about 340 degrees Celsius, and is resistant to thermal degradation, thus making it a desirable material for implantable medical devices. Such thermoplastic materials, however, are not bioactive, osteoproductive, or osteoconductive.

Many implantable devices available today comprise materials that have properties similar to natural bone, such as compositions containing calcium phosphates. Exemplary calcium phosphate compositions contain type-B carbonated hydroxyapatite (Ca₅(PO₄)₃ₓ(CO₃)ₓ(OH)). Calcium phosphate ceramics have been fabricated and implanted in mammals in various forms including, but not limited to, shaped bodies and cements. Different stoichiometric compositions, such as hydroxyapatite (HA), tricalcium phosphate (TCP), tetracalcium phosphate (TTCP), and other calcium phosphate (CaP) salts and minerals have all been employed in attempts to match the adaptability, biocompatibility, structure, and strength of natural bone. Although calcium phosphate based materials are widely accepted, they lack the ease of handling, flexibility and capacity to serve as a liquid carrier/storage media necessary to be used in a wide array of clinical applications. Calcium phosphate materials are inherently rigid, and to facilitate handling are generally provided as part of an admixture with a carrier material; such admixtures typically have an active calcium phosphate ingredient to carrier ratio of about 50:50, and may have as low as 10:90.

A common surgical treatment to repair or replace damaged bone in a patient's body is to implant a fusion device at the location of the damage that can facilitate bone regrowth. For example, specific to the spine, one method of repair is to remove the damaged vertebra (in whole or in part) and/or the damaged disc (in whole or in part) and replace it with an implant or prosthesis. In some cases, it is necessary to stabilize a weakened or damaged spinal region by reducing or inhibiting mobility in the area to avoid further progression of the damage and/or to reduce or alleviate pain caused by the damage or injury. In other cases, it is desirable to join together the damaged vertebrae and/or induce healing of the vertebrae. Accordingly, an implant or prosthesis may be configured to facilitate fusion between two adjacent vertebrae. The implant or prosthesis may be placed without attachment means or fastened in position between adjacent structural body parts (e.g., adjacent vertebral bodies).

Most bone fusion implants are configured mainly to provide a rigid structural framework to support new bone growth at the area to be treated. However, these implants do not necessarily promote new bone growth in and of themselves. Rather, these implants immobilize and/or stabilize the damaged area to reduce further damage. The implants must work in conjunction with an additional bone growth enhancing component to aid in the bone regrowth and/or repair process. For instance, the implants may be coated with a biological agent that promotes bone growth. Quite often, these implants will serve as cages, and include a compartment to hold bone graft material to facilitate fusion.

The role of bone graft materials in clinical applications to aid the healing of bone has been well documented over the years. Most bone graft materials that are currently available, however, have failed to deliver the anticipated results necessary to make these materials a routine therapeutic application in surgery. Improved bone graft materials for forming bone tissue implants that can produce reliable and consistent results are therefore still needed and desired.

In recent years, intensive studies have been made on bone graft materials in the hopes of identifying the key features necessary to produce an ideal bone graft implant, as well as to proffer a theory of the mechanism of action that results in successful bone tissue growth. At least one recent study has suggested that a successful bone tissue scaffold should consider the physicochemical properties, morphology and degradation kinetics of the bone being treated. (“Bone tissue engineering: from bench to bedside”, Woodruff et al., Materials Today, 15(10): 430-435 (2012)). According to the study, porosity is necessary to allow vascularization, and the desired scaffold should have a porous interconnected pore network with surface properties that are optimized for cell attachment, migration, proliferation and differentiation. At the same time, the scaffold should be biocompatible and allow flow transport of nutrients and metabolic waste. Just as important is the scaffold's ability to provide a controllable rate of biodegradation to compliment cell and/or tissue growth and maturation. Finally, the ability to model and/or customize the external size and shape of the scaffold is to allow a customized fit for the individual patient is of equal importance.

Woodruff, et. al. also suggested that the rate of degradation of the scaffold must be compatible with the rate of bone tissue formation, remodeling and maturation. Recent studies have demonstrated that initial bone tissue ingrowth does not equate to tissue maturation and remodeling. According to the study, most of the currently available bone graft materials are formulated to degrade as soon as new tissue emerges, and at a faster rate than the new bone tissue is able to mature, resulting in less than desirable clinical outcomes.

Other researchers have emphasized different aspects as the core features of an ideal bone graft material. For example, many believe that the material's ability to provide adequate structural support or mechanical integrity for new cellular activity is the main factor to achieving clinical success, while others emphasize the role of porosity as the key feature. The roles of porosity, pore size and pore size distribution in promoting revascularization, healing, and remodeling of bone have long been recognized as important contributing factors for successful bone grafting implants. Many studies have suggested an ideal range of porosities and pore size distributions for achieving bone graft success. However, as clinical results have shown, a biocompatible bone graft having the correct structure and mechanical integrity for new bone growth or having the requisite porosities and pore distributions alone does not guarantee a good clinical outcome. What is clear from this collective body of research is that the ideal bone graft material should possess a combination of structural and functional features that act in synergy to allow the bone graft material to support the biological activity and an effective mechanism of action as time progresses.

Currently available bone graft materials fall short of meeting these requirements. That is, many bone graft materials tend to suffer from one or more of the problems previously mentioned, while others may have different, negatively associated complications or shortcomings. One example is autograft implants. Autograft implants have acceptable physical and biological properties and exhibit the appropriate mechanical structure and integrity for bone growth. However, the use of autogenous bone requires the patient to undergo multiple or extended surgeries, consequently increasing the time the patient is under anesthesia, and leading to considerable pain, increased risk of infection and other complications, and morbidity at the donor site.

The roles of porosity, pore size and pore size distribution in promoting revascularization, healing, and remodeling of bone have been recognized as important contributing factors for successful implantable devices. However, currently available materials still lack the requisite chemical and physical properties necessary for an ideal implantable device. For instance, currently available materials tend to resorb too quickly, while some take too long to resorb due to the material's chemical composition and structure. For example, certain materials made from hydroxyapatite tend to take too long to resorb, while materials made from calcium sulphate or B-TCP tend to resorb too quickly. Further, if the porosity of the material is too high (e.g., around 90%), there may not be enough base material left after resorption has taken place to support osteoconduction. Conversely, if the porosity of the material is too low (e.g., 30%) then too much material must be resorbed, leading to longer resorption rates. In addition, the excess material means there may not be enough room left in the residual material for cell infiltration. Other times, the materials may be too soft, such that any kind of physical pressure exerted on them during clinical usage causes them to deform or displace and lose the fluids retained by them.

When it comes to synthetic bone graft substitutes, the most rapidly expanding category consists of products based on calcium sulfate, hydroxyapatite and tricalcium phosphate. Whether in the form of injectable cements, blocks or morsels, these materials have a proven track record of being effective, safe bone graft substitutes for selected clinical applications. Recently, new materials such as bioactive glass (“BAG”) have become an increasingly viable alternative or supplement to polymer-based load bearing implants. In comparison to autograft implants, these new synthetic implants have the advantage of avoiding painful and inherently risky autograft harvesting procedures on patients. Also, the use of these synthetic, non-bone derived materials can reduce the risk of disease transmission. Like autograft and allograft implants, these new artificial implants can serve as osteoconductive scaffolds that promote bone regeneration. Preferably, the implant is resorbable and is eventually replaced with new bone tissue.

Current methods for manufacturing bioactive composites, such as those containing bioactive glass and polymers, suffer from a number of drawbacks. For example, the high reactivity of bioactive materials, such as bioactive glass, with polymers presents a challenge to conventional processing techniques. In particular, the surface alkali of the bioactive materials reacts with the polymer during processing, forming a material that inhibits the proper functioning of certain processing machines that may be used to form the composite device. In addition, this reaction may degrade the functionality and reactivity of the biomaterials and/or cause a degradation of the structural and mechanical properties of the resulting implantable device.

Thus, in order to provide a better clinical solution for the repair and/or replacement of bone, improved bioactive materials, implantable devices, and methods for manufacturing these devices, are needed. It would therefore be desirable to provide an implantable device that combines the benefits of a traditional metal, ceramic or polymer, such as a thermoplastic polymer like PAEK, for mechanical support, but with the benefit of bioactivity, to initiate cellular activity and promote successful bone regeneration. Further, there is also a need in the art for more effective methods for preparing such bioactive composite materials to produce bioactive implants that have the appropriate mechanical properties to withstand the forces required of spinal, orthopedic, dental and other implants. Embodiments of the present disclosure address these and other needs.

### SUMMARY

The present disclosure provides implantable medical devices that are engineered, at least in part, from a composite material comprising a polymer component and a bioactive component incorporated therein to provide bioactivity to the polymer component for the improved treatment of bone or other purposes. These devices are engineered to provide enhanced cellular activity to promote bone fusion and/or regeneration. Composite biomaterials comprising polymer component(s) and bioactive component(s) incorporated therein, methods for making such biomaterials, and methods for making implantable devices from such composite biomaterials, are also provided in this disclosure.

According to one aspect, an implantable device is provided, such as an orthopedic implant, a spinal fusion implant, dental implant, total or partial joint replacement or repair device, trauma repair device, bone fracture repair device, reconstructive surgical device, alveolar ridge reconstruction device, veterinary implant or the like. The implantable device may have a main body comprising a polymeric framework, and a bioactive material additive incorporated into the polymeric framework. In some embodiments, the bioactive material additive is incorporated substantially throughout the polymeric framework. Incorporating the bioactive material additive substantially throughout the polymeric framework provides cellular activity through the interior of the implantable device, rather than just on its surface, thereby further enhancing and accelerating bone growth and induction.

In addition, with certain unique manufacturing techniques of the present disclosure (discussed below), the implantable device may include multiple combinations of composite materials. For example, the implantable device may include certain portions with different percentages of bioactive materials and polymer materials to provide for staged resorption, increased mechanical strength and/or to enhance bioactivity in certain areas of the device.

The polymer may comprise any suitable polymer for use in an implantable device, including but not limited to, a polyalkenoate, polycarbonate, polyamide, polyether sulfone (PES), polyphenylene sulfide (PPS), or a polyaryletherketone (PAEK), such as polyetheretherketone (PEEK) or polyetherketoneketone (PEKK). In other embodiments, the polymer may comprise a bioresorbable material, such as polyglycolic acid (PGA), poly-l-lactic acid (PLLA), poly-d-lactic acid, polycyanoacrylates, polyanhydrides, polypropylene fumarate and the like. The bioresorbable material may comprise all or only a portion of the polymer component and may, for example, be mixed or combined with a non-resorbable polymer.

The bioactive material additives of the present disclosure may be in the form of frit, fibers, pellets, powder, microspheres, granules or other particles that are mixed with frit, fibers, pellets, powder, granules, microspheres or other particles of the polymer to form a bioactive composite. For the sake of convenience, the term “particles” shall be defined herein as frit, fibers, powder, granules, pellets, microspheres or the like. The bioactive material may comprise fused particles, morsels or porous granules, such as porograns, which are highly porous granular spherical particles that typically have larger surface areas available for cellular activity. The bioactive composite may be further processed and/or combined with the main body into a shaped implantable device having the appropriate properties to withstand the forces required of the implant.

The bioactive material additives may include silica-based materials, boron-based materials and/or strontium-based materials or any combinations thereof. The bioactive material may be glass-based, ceramic-based, a hybrid glass ceramic material that is partially amorphous and partially crystalized or a combination thereof. For example, the bioactive material additive may include one or more of sol gel derived bioactive glass, melt derived bioactive glass, silica based bioactive glass, silica free bioactive glass such phosphate based bioactive glass, crystallized bioactive glass (either partially or wholly), and bioactive glass containing trace elements or metals such as copper, zinc, strontium, magnesium, zinc, fluoride, mineralogical calcium sources, strontium and/or boron-based bioactive materials, such as borate. In certain embodiments, the bioactive glass comprises 45S5 bioactive glass, Combeite and/or a boron-based bioactive material, or a mixture thereof.

In certain embodiments, the bioactive material may be coated with certain materials. The bioactive material may be silanated or silanized, such that its surface is substantially covered with organofunctional alkosilane molecules. Suitable organofunctional alkosilane molecules includes, but are not limited to, aminosilanes, glycidoxysilanes, mercaptosilanes and the like. Silanization of the bioactive materials increases its hydrophobicity and may create a chemical bond that increases it mechanical strength. In addition, silianization of the bioactive material increases the overall pH of the material, thereby slowing down degradation and potentially controlling the resorption rate

The average diameter of the bioactive material can be between about 0.1 to about 2,000 microns. In exemplary embodiments, the average diameter of the bioactive material can be between about 0.1 and about 400 microns, or about 50 to about 200 microns.

In another aspect, the implantable device may comprise a main body and a bioactive component that includes a polyarylretherketone (PAEK) polymer component and a bioactive additive component incorporated substantially throughout the polymer component. In some embodiments, the main body may comprise a polymer, a metal, a ceramic, a bioactive composite, or any combination thereof.

The polymer component may comprise polyetheretherketone (PEEK), polyetherketoneketone (PEKK) or a mixture thereof. While these materials include excellent mechanical properties, particularly for load-bearing implants, they are not bioactive or osteoconductive. Thus, providing a device that includes a bioactive additive fully integrated substantially throughout the polymer component provides a number of distinct advantages. In particular, these devices provide enhanced cellular activity throughout substantially the entire implantable device which further promotes bone fusion and/or regeneration.

The average diameter of the PAEK polymer can be between about 0.5 microns to about 4,000 microns. The average diameter may be less than 1,000 microns. In other embodiments, the average diameter of the PAEK polymer can be greater than 400 microns. In certain embodiments, the average diameter of the PAEK polymer can be between 400 to 1,000 microns. This particle size is suitable for compounding with bioactive and boron-based glasses having a particle, pellet or fiber size of 0.1-200 microns.

The main body of the implantable device may include an outer surface having a non-smooth, roughened surface. This roughened surface may be achieved by subjecting the bioactive composite to secondary processing techniques to increase the surface area of the device. These secondary processing techniques may, for example, include sanding or otherwise roughening the outer surface of the main body after it has been formed. In certain embodiments, the secondary processing may include grit blasting all, or a portion of, the surface of the implantable device. The bioactive materials of the present disclosure may be used as the media for grit blasting the surface of the device.

Applicant has discovered that sanding (or otherwise machining) the surface of the bioactive composite device after its formation results in significant bioactivity at substantially the entire surface that is machined. Sanding or otherwise machining the surface may expose particles or micropores within the material that are below the outer surface to allow bone tissue to grow into the main body and/or it may draw the bioactive materials to the surface of the device. In addition, sanding the surface increases the overall surface area of the composite device by creating a rougher surface that has more surface area to interact with bone tissue.

The main body may be formed as a rigid framework and the bioactive component may be incorporated into, or on, at least a portion of the rigid framework. In certain embodiments, the main body comprises an outer surface and the bioactive component may be disposed on, or around, at least a portion of this outer surface. The bioactive component may be disposed on substantially the entire outer surface of the main body. The bioactive component may form one or more layers disposed adjacent to, or between, one or more layers of the main body.

In other embodiments, the main body may comprise one or more chambers, pores or other internal spaces and the bioactive component may be disposed adjacent to, or within, these internal spaces. In certain embodiments, the bioactive component may comprise one or more bundles of particles disposed within, or on, the main body.

The bioactive component may be incorporated, or otherwise embedded, throughout the main body. The main body and the bioactive component may be formed from particles mixed together into a substantially homogenous composite such that the overall implantable device has substantially the same properties throughout. Alternatively, the bioactive component and the main body may be non-homogenous such that the bioactive component is interspersed throughout the main body.

In certain embodiments, the main body and the bioactive component are both made from a thermoplastic polymer, such as PAEK, and bioactive particles. The bioactive particles may be mixed with the polymer particles to form a substantially homogenous composite that is processed through, for example, compression molding or extrusion to shape the implantable device.

The implantable device may include different bioactive materials that each have a different resorption capacity. In some embodiments, the weight ratio and/or the particle size ratio of the bioactive particles are selected to enable staged resorption of the bioactive particles within the body. The resorption rate of a fiber is determined or controlled by its material composition and by its diameter. The material composition may result in a slow reacting vs. faster reacting product. For example, certain compositions of the bioactive particles may resorb more quickly than others (e.g., boron-based particles typically resorb more quickly than silica-based bioactive glass particles). The weight ratio, crystallinity and/or the particle size ratio of the boron-based particles and the bioactive glass particles are selected to enable a staged resorption of both particles, thereby ensuring that the implant withstands loads within the body while enhancing the cellular activity that promotes bone growth and/or fusion/interdigitation of the bone and tissue within the implant.

In certain embodiments, the ratio of weight of the various bioactive materials in the device is selected to provide staged resorption of these particles within the body. In other embodiments, the ratio of a particle size of the bioactive glass to a particle size of the boron-containing bioactive particles is selected to provide staged resorption within the body.

The implantable device may be a custom device that is designed for an individual patient's specific anatomy. The size and shape of the implantable device may be based, for example, on patient CT scans, MRIs or other images of the patient's anatomy, In certain embodiments, these images may be used to form a customized device through additive manufacturing techniques, such as stereolithography (SLA), selective layer melting (SLM), selective laser sintering (SLS), E-beam or 3D printing of metal, metal alloy or polymer, and fused deposition modeling (FDM). In other embodiments, the images may be used to create molds for forming the customized device.

The implantable device may be porous or nonporous. The pore sizes may be uniform or variable throughout the implantable device.

The implantable device may comprise a lattice structure. The lattice structure may include a framework formed from a metal, polymer or ceramic with a bioactive component. The lattice structures of the present disclosure may include repeating units of geometric structure, or they may be formed with random geometric structures throughout the lattice. These porous lattice structures provide room for osseointegration by providing a scaffold to encourage cell on-growth and in-growth into the pore spaces. The empty spaces within the lattice allow for fluids and nutrients to enter the implant, thereby allowing for osteointegration of bone tissue.

The lattice structure itself may be created in-vivo with bioactive or resorbable materials that either dissolve or assimilate into the bone tissue. In certain embodiments, the lattice structure implants may be engineered to incorporate two separate phases in-vivo. In the first phase, fluids and nutrients are allowed to pass into the empty spaces of the lattice to provide for osteointegration. In the second phase, the actual lattice framework may be formed completely or partially from resorbable materials (as discussed above) such that the entire, or at least a portion of, the structure dissolves, thereby leaving only bone tissue behind.

The device may be porous and/or bioresorbable, and may be configured to be load-bearing. The device may be non-porous. In addition, the device may include a biological agent. The biological agent may be selected from and is not limited to the group consisting of glycosaminoglycans, growth factors, synthetic factors, recombinant factors, allogenic factors, stem cells, demineralized bone matrix (DBM), or cell signaling agents.

In certain embodiments, the bioactive component comprises fibers or other particles and the main body comprises pores. The pores may extend in a direction substantially parallel to the fibers or particles. The pores may extend along a length of the fibers or particles. The main body has a first surface and a second surface opposite the first surface. The pores preferably extend from the first surface to the second surface. In certain embodiment, the fibers and/or pores may form one or more tubes extending from the first surface to the second surface.

The bioactive fibers or other particles may be directionally aligned with each other to enhance and direct the growth of tissues through the main body from the first surface to the second surface to ultimately improve the mechanical bond between the implant the surrounding tissues. The pores present in the directional fiber assemblage will promote the migration of hard and soft tissue in the spaces between the fibers. The bioactive particles may be randomly aligned to provide multi-directionality.

In one embodiment, the fibers or other particles comprise a material configured to promote the circulation of liquids between the fibers. The particles may be configured to promote capillary action between aligned fibers to pull fluids therethrough. This constant movement of fluids will enhance tissue growth as oxygen and nutrients are brought into the implant and metabolic waste products are removed. This capillary action will continue indefinitely until the fibers are filled with new tissue and the forces between body fluids and the pore volume are eliminated.

The aligned porosity can also enhance the dispersion or absorption of materials such as bone marrow aspirate that are often added to promote healing in load bearing implants prior to implantation. The capillary action of the aligned fibers pulls the cells and body fluids present in the marrow through the assemblage to start the healing process.

In another aspect, an implantable device is provided that comprise a plurality of compressed bioactive glass fibers. In some embodiments, the device may further comprise a plurality of bioactive glass particulates. The bioactive glass fibers may be randomly oriented, or may be aligned with respect to one another. In order to provide a load-bearing device, the fibers can be sintered together. The device may comprise a plurality of bundles of compressed bioactive glass fibers within the main body. The plurality of bundles of compressed bioactive glass fibers may be equidistantly spaced apart from one another within the main body. The device may be shaped as a cylinder. The device may be porous, or bioresorbable.

The fiber bundles may be incorporated into a composite implantable device. In such a design, the fiber bundles may be at least partially, if not fully, contained within a main body of the implantable device and selectively aligned relative to the device to provide directionality of cell growth through the device. The fiber bunders may be uniformly aligned with each other, or they may be aligned in different directions relative to each other. For example, the fiber bundles can extend along one or more axes of the implantable device to provide cell growth along those axes. In another example, the fiber bundles may be randomly oriented relative to each other, but selectively aligned relative to the implantable device. In all of these examples, the main body of the implantable device may include a polymer with bioactive materials incorporated throughout the polymer according to any of the embodiments disclosed herein.

In another aspect, the implantable device may be engineered to allow for bone growth in specific directions or dimensions. The device may be designed with an anchorage point with telescoping capability into different planes. This allows the device to be compatible with, for example, bones that are still growing in children or young adults.

In another aspect of the invention, an implantable device comprises a rigid body formed from a bioactive composite material comprising a polymer component and a bioactive glass additive component incorporated throughout the polymer component. Each of the polymer component and additive component are in the form of particles. The average particle sizes of the polymer component and the additive component may be matched, i.e., substantially the same. In other embodiments, the average particle sizes of the polymer component are different from the average particle sizes of the additive component, and selected for mechanical strength or processing purposes. The particle sizes may also be selected to enable staged resorption of the bioactive glass component within the patient's body.

The polymer may comprise a polyalkenoate, polycarbonate, polyamide, polyether sulfone (PES), polyphenylene sulfide (PPS), or a polyaryletherketone (PAEK) like polyetheretherketone (PEEK) or polyetherketoneketone (PEKK) or a mixture thereof. In certain embodiments, the polymers comprise polyetheretherketone (PEEK) or polyetherketoneketone (PEKK). In certain embodiments, the polymer may comprise a bioresorbable material, such as polyglycolic acid (PGA), poly-l-lactic acid (PLLA), poly-d-lactic acid, polycyanoacrylates, polyanhydrides, polypropylene fumarate and the like. The bioresorbable material may comprise all or only a portion of the polymer component and may, for example, be mixed or combined with a non-resorbable polymer.

The bioactive additive may be in the form of frit, fibers, powder, granules, pellets, microspheres or other particles that are mixed with frit, fibers, powder, granules, pellets, microspheres or other particles of the polymer to form a substantially homogenous bioactive composite that is further processed into a shaped implantable device having the appropriate properties to withstand the forces required of the implant. The polymer particles and the bioactive particles are mixed together without using a solvent to form a dispersion or remove/reduce the alkalinity of the bioactive material. The bioactive particles may also be mixed with the polymer particles, fibers or pellets without the need to pre-heat the inert polymer prior to processing.

The bioactive material additive may include silica-based materials, boron-based materials and/or strontium-based materials or any combinations thereof. The bioactive material may be glass-based, ceramic-based, a hybrid glass ceramic material that is partially amorphous and partially crystalized or a combination thereof. For example, the bioactive material additive may include one or more of sol gel derived bioactive glass, melt derived bioactive glass, silica based bioactive glass, silica free bioactive glass such phosphate based bioactive glass, crystallized bioactive glass (either partially or wholly), and bioactive glass containing trace elements or metals such as copper, zinc, strontium, magnesium, zinc, fluoride, mineralogical calcium sources, strontium and/or boron-based bioactive materials, such as borate. In certain embodiments, the bioactive glass comprises 45S5 bioactive glass, Combeite and/or a boron-based bioactive material, or a mixture thereof.

In some embodiments, the average diameter of the bioactive glass and/or the boron-based material is between about 0.1 to about 2,000 microns. In exemplary embodiments, the average diameter of the bioactive glass and/or the boron-based material is between about 0.1 and about 400 microns, or about 50 to about 200 microns.

The implantable device may be an orthopedic implant, a spinal fusion implant, dental implant, total or partial joint replacement or repair device, trauma repair device, bone fracture repair device, reconstructive surgical device, alveolar ridge reconstruction device, or veterinary implant. In certain embodiments, the device has a shape and geometry configured for insertion between adjacent bone segments, such as vertebral bodies to facilitate bone fusion.

In another aspect of the present disclosure, various processes for forming an implantable device from a bioactive composite polymeric material are provided.

In certain aspects, the implantable device may be formed by an additive manufacturing technique whereby layers of material are formed and then deposited on each other to create the final device. These additive manufacturing techniques may include stereolithography (SLA), selective layer melting (SLM), selective laser sintering (SLS), E-beam or 3D printing of metal, metal alloy or polymer, fused deposition modeling (FDM) or combinations.

In these embodiments, the layers of material that are deposited onto each other may each have different concentrations of bioactive glass. This provides for different levels of bioactivity and/or resorption within different portions of the resulting implantable device. In certain embodiments, the outer layers of the polymer may have greater concentrations of bioactive additive than the inner layers such that the outer layers react with bone tissue more quickly than the inner layers. This design creates relatively rapid bioactivity on the outer layers and a longer and slower bioactivity throughout the interior of the device.

In certain embodiments, for example, one or more of the outer layer(s) of the polymer component may have a concentration of about 0-100 percent bioactive additive and 0-100 percent polymer; whereas the inner layers may have a concentration of about 0-100 percent bioactive additive and about 0-100 percent polymer. In one such example, the outer layer comprises about 40% to 100% bioactive glass and about 0% to about 60% polymer and the interior comprises about 5% to about 40% bioactive material additive and about 60% to about 95% polymer. In another example the outer surface may comprise about 75% to about 100% bioactive material additive and about 0% to about 25% polymer and the inner portions comprises about 5% to about 25% bioactive material additive and about 75% to about 95% polymer.

In other aspects, the process includes mixing particles, fibers or pellets of a polyaryletherketone (PAEK) polymer and a bioactive additive to form a substantially homogenous mixture. Substantially homogenous according to the present disclosure means that the mixture is substantially uniform with substantially the same properties throughout. The mixture is then compressed and heated to at least the melting temperature of the individual polymer to form a bioactive composite in a shape of the load bearing implantable device.

The methods disclosed herein take advantage of injection and/or compression molding techniques such that the polymer and the bioactive material may be inserted into the mold in the form of powder, fibers, pellets or other particles that have been readily metered by weight. Composite pellets may be used as input for compression molding techniques. For the purpose of this example, composite pellets means pellets containing bioactive materials and polymer material mixed together. This has the advantage that the bioactive material is mixed with the polymer to produce a substantially homogenous bioactive composite. The polymer particles, fibers or pellets and the bioactive particles or fibers are preferably mixed together without using a solvent to remove the alkalinity of the bioactive material. The bioactive particles or fibers may also be mixed with the polymer particles, fibers or pellets without the need to pre-heat the inert polymer prior to processing.

In certain embodiments, the bioactive composite device may be subjected to secondary processing techniques to increase the surface area of the device. Applicant has discovered that sanding (or otherwise machining) the surface of the bioactive composite device after its formation results in significant bioactivity around substantially the entire surface of the device. Sanding or otherwise machining the surface draws the bioactive materials to the surface of the device. In addition, sanding the surface increases the overall surface area of the composite device by creating a rougher surface that has more surface area to interact with bone tissue.

In certain embodiments, the particles of PAEK polymer and bioactive additive are in the form of a powder. The bioactive additive may comprise a bioactive glass and/or a boron-based bioactive material. The boron-based bioactive material may comprise borate. The bioactive glass may comprise any suitable bioactive glass, such as Combeite, 45S5 bioactive glass or a combination thereof.

The PAEK polymer particles, pellets or fibers may have an average diameter of about 0.5 to about 4,000 microns. The average diameter may be about 400 to about 1,000 microns. In some embodiments, the average diameter is about 45 microns to about 65 microns. The borate particles and the bioactive glass may have an average diameter of about 0.1 to about 2,000 microns, or between about 0.1 and about 400 microns, or about 50 to about 200 microns. In some embodiments, the average diameter is about 90 microns to about 355 microns.

In another aspect of the invention, a load bearing implantable device is formed through the process described above. The load bearing implantable device may be porous.

In another aspect of the invention, a process for forming a load bearing implantable device comprises mixing particles, pellets or fibers of a polyaryletherketone (PAEK) polymer and a bioactive additive into a screw extruder, rotating the screw extruder and heating the particles of the PAEK polymer and the bioactive additive to at least a melting temperature of the particles to form a substantially homogenous composite in a shape of the load bearing implantable device.

Extrusion devices that can be employed, for example, include single and twin-screw machines, co-rotating or counterrotating, closely intermeshing twin-screw compounders and the like. In one embodiment, the screw extruder may be a twin screw extruder with two meshing screws that are commonly used to plasticize and extrude plastic materials.

In certain embodiments, the PAEK polymer and the bioactive additive are in the form of a powder. The bioactive additive may comprise bioactive glass, such as 45S5 or Combeite and/or boron-based material, such as borate. The process includes mixing the powders of the PAEK polymer and the bioactive additive together to form a substantially homogenous mixture and then placing the homogenous mixture into the screw extruder.

In another embodiment, the PAEK polymer is in the form of pellets and the bioactive additive is in the form of powder. The PAEK pellets are first inserted into the screw extruder and rotated and heated until the pellets form into a melted plastic. The bioactive powder is then mixed into the extruder with the PAEK material to form a homogenous product. This homogenous product is then further rotated and heated to form a bioactive composite that can be shaped into a load bearing implant.

In another aspect of the invention, a load bearing implantable device is formed through the process described above.

In yet another aspect of the invention, a method for forming a load bearing implantable device includes mixing particles of a polyaryletherketone (PAEK) polymer and a bioactive additive into a screw extruder and rotating the screw extruder to form homogenous composite pellets. The pellets are then compressed and heated to at least a melting temperature of the pellets to form a bioactive composite in a shape of the load bearing implantable device.

In this embodiment, homogenous pellets are formed that can be re-processed and compression or injection molded into the desired shape.

In another aspect of the invention, a load bearing implantable device is formed through the process described above.

In yet another aspect of the invention, a method for forming an implantable device comprises placing polymer and bioactive material powder, pellets or other particles into a compression molder and/or a screw extruder (single, twin, etc.) to produce composite pellets or other shapes. These composite pellets/shapes are then injection molded into a desired shape. The resulting product may be subjected to secondary processing comprising sanding or other machining to increase surface exposure of bioactive glass.

In still another aspect of the invention, the polymer and bioactive material may be extruded by screw extruder (single, twin, etc.) into filaments of composite bioactive polymeric material. These composite bioactive polymeric filaments may then be further processed into a final, shaped implantable device. For example, the filaments may be fed into a 3D printer to provide a final product. One such technique would involve 3D printing the composite filaments using fused deposition modeling (FDM) to form the desired product.

It is to be understood that both the foregoing general description and the following detailed description are exemplary and explanatory only and are not restrictive of the disclosure. Additional features of the disclosure will be set forth in part in the description which follows or may be learned by practice of the disclosure.

The foregoing and other features of the present disclosure will become apparent to one skilled in the art to which the present disclosure relates upon consideration of the following description of exemplary embodiments.

### BRIEF DESCRIPTION OF THE DRAWINGS

The accompanying drawings and photographs, which are incorporated in and constitute a part of this specification, illustrate several embodiments of the disclosure and together with the description, serve to explain the principles of the disclosure.

FIG. 1 illustrates an example of an implantable device having a main body with a bioactive component around its outer surface according to certain embodiments of the present disclosure;

FIG. 2 illustrates an example of an implantable device having a main body with a bioactive component on certain surfaces of the main body;

FIG. 3 illustrates an example of a porous implantable device according to the present disclosure;

FIG. 4 illustrates an example of an implantable device having a main body with a bioactive component incorporated therein;

FIG. 5 illustrates an example of an implantable device having a bioactive component layer within one or more layers of a main body according to the present disclosure;

FIG. 6 illustrates an example of an implantable device comprising a cage component and a bioactive component contained therein;

FIG. 7 illustrates an example of an implantable device formed with directionally-aligned bioactive components;

FIG. 8A illustrates an implantable device comprising a plurality of bundles of uniformly aligned bioactive components;

FIG. 8B illustrates an implantable device comprising a plurality of bundles of randomly aligned bioactive components;

FIG. 9 illustrates a composite implantable device comprising a cage component and a bone graft component;

FIG. 10 illustrates a composite implantable device comprising a multi-part cage opponent and a bone graft component;

FIG. 11 illustrates a cross-sectional view of a composite implantable device comprising a cage component and different bone graft components associated therewith;

FIG. 12 illustrates another composite implantable device comprising a cage component and a bone graft component contained therein.

FIGS. 13A and 13B illustrate examples of an implantable device incorporating directionally aligned bioactive components;

FIGS. 14A and 14B are photographic images of an implantable device formed with directionally aligned bioactive components;

FIG. 15 is a magnified photographic image of an implantable device having an additional bioactive coating on its outer surface;

FIG. 16 is a magnified photographic image of an implantable device, illustrating pores for cellular attachment to a bioactive component;

FIGS. 17A-17C illustrate examples of lattice structures including a main body framework having a bioactive component incorporated therein according to the present disclosure;

FIGS. 18A-18E illustrate various examples of shapes for individual units forming a lattice structure of an implant according to the present disclosure;

FIGS. 19 and 20 illustrate examples of implantable cervical fusion implants comprising a polymer and having incorporated therein a bioactive component according to the present disclosure;

FIGS. 21 and 22 illustrate examples of interbody fusion implants comprising a polymer and having incorporated therein a bioactive component according to the present disclosure;

FIG. 23 illustrates an example of a cervical plate comprising a polymer and having incorporated therein a bioactive component according to the present disclosure;

FIGS. 24 and 25 illustrate examples of artificial discs comprising a polymer and having incorporated therein a bioactive component according to the present disclosure;

FIG. 26 illustrates an example of an artificial hip implant comprising a polymer and having incorporated therein a bioactive component according to the present disclosure;

FIG. 27 illustrates an example of an artificial knee implant comprising a polymer and having incorporated therein a bioactive component according to the present disclosure;

FIG. 28 illustrates an example of a fracture plate for a wrist comprising a polymer and having incorporated therein a bioactive component according to the present disclosure;

FIG. 29 illustrates an example of a bone dowel comprising a polymer and having incorporated therein a bioactive component according to the present disclosure;

FIGS. 30A-30C illustrate various examples of bone anchors comprising a polymer and having incorporated therein a bioactive component according to the present disclosure;

FIGS. 31 and 32 illustrate examples of maxillofacial implants comprising a polymer and having incorporated therein a bioactive component according to the present disclosure;

FIG. 33 illustrates an example of a cranial implant comprising a polymer and having incorporated therein a bioactive component according to the present disclosure;

FIG. 34 is a photographic image of an exemplary load bearing implantable device formed in accordance with a process of the present disclosure;

FIGS. 35A and 35B are photographic images taken at different magnifications (20×, 40×, respectively) showing the bioactivity of an implantable device at seven days having 20% by weight 45S5 bioactive glass, without sanding the device;

FIGS. 36A and 36B are photographic images taken at different magnifications (20×, 40×, respectively) showing the bioactivity of an implantable device at seven days having 20% by weight 45S5 bioactive glass after sanding the device;

FIGS. 37A and 37B are photographic images taken at different magnifications (20×, 40×, respectively) showing the bioactivity of an implantable device at thirty-four days having 20% by weight 45S5 bioactive glass without sanding the device;

FIGS. 38A and 38B are photographic images taken at different magnifications (20×, 40×, respectively) showing the bioactivity of an implantable device at thirty-four days having 20% by weight 45S5 bioactive glass after sanding the device;

FIGS. 39A and 39B are photographic images taken at different magnifications (20×, 40×, respectively) showing the bioactivity of an implantable device at seven days having 20% by weight boron-based particles without sanding the device;

FIGS. 40A and 40B are photographic images taken at different magnifications (20×, 40×, respectively) showing the bioactivity of an implantable device at seven days having 20% by weight boron-based particles after sanding the device;

FIGS. 41A and 41B are photographic images taken at different magnifications (20×, 40×, respectively) showing the bioactivity of an implantable device at thirty-four days having 20% by weight boron-based particles without sanding the device;

FIGS. 42A and 42B are photographic images taken at different magnifications (20×, 40×, respectively) showing the bioactivity of an implantable device at thirty-four days having 20% by weight boron-based particles after sanding the device;

FIG. 43 illustrates an example of a composite material or implantable device having an inner core surrounded by an outer portion, each having a different percentage of bioactive material incorporated within a polymer; and

FIGS. 44A and 44B are graphs illustrating the viscosity over time for certain mixtures of polymer and bioactive materials in a parallel plate rheometer.

### DETAILED DESCRIPTION

When it comes to orthopedic biomaterials, hard materials such as metals and ceramics primarily come to mind. This is particularly the case with load-bearing orthopedic applications. However, recent advancements in polymer science and technology have allowed certain polymers and composites to not only be viable, but preferable, alternatives to more traditional metal and ceramic biomaterials. In bearing and wear applications, polymers provide advantages over metals by being lighter and having lower frictional properties compared to metals. These polymeric materials can withstand repeated friction and wear for high-load applications, yet can still match the strength of metals.

Additionally, polymers are biocompatible and are more resistant to chemicals than their metal counterparts, which is a benefit during certain high precision manufacturing process as many of these techniques involve harsh and/or corrosive chemicals that would negatively affect metallic materials. Polymers are also resistant to impact damage, making them less prone to denting or cracking the way metals do.

A certain group of polymers, the polyaryletherketones (PAEK), which includes polyetheretherketone (PEEK) and polyetherketoneketone (PEKK), has shown great promise as a biomaterial for having mechanical properties similar to human bone tissue, a lack of electrochemical activity in vivo, excellent corrosion resistance and biocompatibility, considerable fatigue strength, wear resistance, tensile strength, compressive strength, and ductility. With a favorable elastic modulus, stress shielding that is often a drawback observed with titanium and titanium alloy is avoided. All of these superior characteristics that are possessed by PEEK and PEKK can be further enhanced by combining it with other additives to lend it bioactivity.

Accordingly, the present disclosure provides various bioactive composite materials and implantable devices that are engineered as a composite device comprising a polymer, such as a thermoplastic polymer, with a bioactive additive, for the improved treatment of bone. The present disclosure also provides methods of manufacturing bioactive composite materials and devices formed from such bioactive composite materials. These devices are engineered to provide enhanced cellular activity to promote bone fusion or regeneration, while providing sufficient structural integrity to support the fusion or regeneration of bone tissue.

In certain aspects, the implantable devices may be engineered, at least in part, with a polymer component, and a bioactive component, for the improved treatment of bone and other purposes. These devices are engineered to provide enhanced cellular activity to promote bone fusion and/or regrowth into, or around, the implantable device. The implantable device may be an orthopedic implant, a spinal fusion implant, dental implant, total or partial joint replacement or repair device, trauma repair device, bone fracture repair device, reconstructive surgical device, alveolar ridge reconstruction device, veterinary implant or the like.

In certain aspects, the implantable devices may be implantable fusion devices. Unlike conventional implantable fusion devices that require an additional bone graft component to provide the devices with bioactivity, the engineered composite fusion devices have the bioactive additive incorporated into the devices themselves. There is no requirement for a separate bone graft component and a separate metal or polymer fusion cage component; both components can be incorporated into the composite implantable fusion devices.

The polymer component may comprise any suitable polymer material for use in load or non-load bearing implantable devices, including but not limited to, polyalkenoate, polycarbonate, polyamide, polyether sulfone (PES), polyphenylene sulfide (PPS), or polyaryletherketone (PAEK), such as polyetheretherketone (PEEK) or polyetherketoneketone (PEKK) or a mixture thereof. In certain embodiments, the polymers comprises polyetheretherketone (PEEK) or polyetherketoneketone (PEKK). In other embodiments, the polymer may comprise a bioresorbable material, such as polyglycolic acid (PGA), poly-l-lactic acid (PLLA), poly-d-lactic acid, polycyanoacrylates, polyanhydrides, polypropylene fumarate and the like. The bioresorbable material may comprise all or only a portion of the polymer component and may, for example, be mixed or combined with a non-resorbable polymer.

The present disclosure also provides methods for manufacturing implantable devices that include a polymeric framework with a bioactive additive incorporated therein. Recent advancements in manufacturing techniques, particularly additive manufacturing techniques and rapid prototyping techniques, such as stereolithography (SLA), selective layer melting (SLM), selective laser sintering (SLS), E-beam or 3D printing of metal, metal alloy or polymer, and fused deposition modeling (FDM) have provided the medical device field exciting new opportunities to create complex metal structures with intricate microstructures not possible before. In addition, combinations of materials can now be integrated together during manufacturing to form unique composite devices. The engineered composite fusion devices of the present disclosure take advantage of these newly developed manufacturing techniques.

Stereolithography or SLA is an additive manufacturing process that, in its most common form, works by focusing an ultraviolet (UV) laser onto a vat of photopolymer resin. With the help of computer aided manufacturing or computer-aided design (CAM/CAD) software, the UV laser is used to draw a pre-programmed design or shape on to the surface of the photopolymer vat. Photopolymers are sensitive to ultraviolet light, so the resin is photochemically solidified and forms a single layer of the desired 3D object. Then, the build platform lowers one layer and a blade recoats the top of the tank with resin. This process is repeated for each layer of the design until the 3D object is complete. Completed parts must be washed with a solvent to clean wet resin from their surfaces.

The present disclosure also provides methods for manufacturing implantable devices that include a polymer, such as PAEK, and a bioactive component. The methods of the present disclosure mix particles of the polymer and the bioactive materials into a substantially homogenous composite. The particles may be frit, pellets, granules, powder, fibers, microspheres or the like. The methods of the present disclosure may allow for particles of the PAEK and the bioactive component to have different or mis-matched particle sizes prior to mixing them to form the homogenous composite. In addition, the composite device may be prepared without the use of a solvent to remove the alkalinity of the bioactive material.

The methods of the present disclosure also allow for the preparing of the bioactive composite without preheating the polymer prior to processing. In addition, the bioactive composite may be prepared in large batches that can be further processed to product shaped implants that have the appropriate mechanical properties to withstand the forces required of spinal, orthopedic, dental or other implants.

The implantable devices of the present disclosure can generally be categorized as either a self-contained, or standalone, implantable device that comprises a main body and a bioactive component. The main body may comprise a polymer, such as PEAK, a metal, a ceramic, a combination of any of these materials, or another suitable material depending on the desired functions of the implantable device. The bioactive component may comprise a polymer component, such as polyetheretherketone (PEEK), polyetherketoneketone (PEKK) or a mixture thereof along with other polymers and additives. The bioactive additive component further comprises at least a bioactive glass and/or a boron-containing bioactive material.

The main body of the implantable device may include an outer surface having a non-smooth, roughened surface. This roughened surface may be achieved by subjecting the bioactive composite to secondary processing techniques to increase the surface area of the device. These secondary processing techniques may, for example, include sanding or otherwise roughening the outer surface of the main body after it has been formed. In certain embodiments, the secondary processing may include grit blasting all, or a portion of, the surface of the implantable device. The bioactive materials of the present disclosure may be used as the media for grit blasting the surface of the device.

Applicant has discovered that sanding (or otherwise machining) the surface of the bioactive composite device after its formation results in significant bioactivity at substantially the entire surface that is machined. Sanding or otherwise machining the surface may expose particles or micropores within the material that are below the outer surface to allow bone tissue to grow into the main body and/or it may draw the bioactive materials to the surface of the device. In addition, sanding the surface increases the overall surface area of the composite device by creating a rougher surface that has more surface area to interact with bone tissue.

The implantable devices may be subject to other secondary processes, such as heat treatment processes. In one such process, the devices are annealed to alter the physical and/or chemical properties of the material to increase its ductility and reduce its hardness marking it more workable. This process involves heating a material above its recrystallization temperature, maintaining a stable temperature for an appropriate amount of time and then cooling. Atoms migrate in the crystal lattice and the number of dislocations decreases leading to a change in ductility and hardness. As the material cools, it recrystallizes.

Applicant has discovered that annealing the composite materials of the present disclosure can modify the crystallinity of the device to homogenize the material, remove irregularities, reduce the inner stresses, increase ductility, increase toughness and agility, improve the material structure, reduce hardness and brittleness, improve the magnetic properties and improve the overall appearance of the device.

The standard method for healing natural tissue with synthetic materials has been to provide a device having the microstructure and macrostructure of the desired end product. Where the desired end product is cancellous bone, traditional bone grafts have been engineered to mimic the architecture of cancellous bone. Although this has been the current standard for bone grafts, it does not take into account the fact that bone is a living tissue. Each bony trabeculae is constantly undergoing active biologic remodeling in response to load, stress and/or damage. In addition, cancellous and cortical bone can support a vast network of vasculature. This network not only delivers nutrients to sustain the living environment surrounding bone, but also supports red blood cells and marrow required for basic biologic function. Therefore, merely providing a synthetic material with the same architecture that is non-biologic is insufficient for optimal bone healing and bone health. Instead, what is required is a mechanism that can recreate the living structure of bone.

Traditional synthetics act as a cast, or template, for normal bone tissue to organize and form. Since these synthetics are not naturally occurring, eventually the casts or templates have to be resorbed to allow for normal bone to regenerate. If these architectured synthetics do not resorb and do not allow proper bone healing, they simply become foreign bodies that are not only obstacles, but potentially detrimental, to bone healing. This phenomenon has been observed in many studies with slow resorbing or non-resorbing synthetics. Since these synthetics are just inert, non-biologic structures that only resemble bone, they behave as a mechanical block to normal bone healing and development.

With the understanding that bone is a living biologic tissue and that inert structures will only impede bone healing; a different physiologic approach is presented with the present disclosure. Healing is a phasic process starting with some initial reaction. Each phase builds on the reaction that occurred in the prior phase. Only after a cascade of phases does the final development of the end product occur—bone. The traditional method has been to replace or somehow stimulate healing by placing an inert final product as a catalyst to the healing process. This premature act certainly does not account for the physiologic process of bone development and healing.

The physiologic process of bone healing can be broken down to three phases: (a) inflammation; (b) osteogenesis; and (c) remodeling. Inflammation is the first reaction to injury and a natural catalyst by providing the chemotactic factors that will initiate the healing process. Osteogenesis is the next phase where osteoblasts respond and start creating osteoid, the basic material of bone. Remodeling is the final phase in which osteoclasts and osteocytes then recreate the three-dimensional architecture of bone.

Bioactive materials of the implantable fusion devices attempt to recapitulate the normal physiologic healing process by presenting the fibrous structure of the fibrin clot. Since the bioactive particles are both osteoconductive as well as osteostimulative, the fibrous network within the composite implantable fusion devices will further enhance and accelerate bone induction. Further, the free-flowing nature of the bioactive matrix or scaffold allows for natural initiation and stimulation of bone formation rather than placing a rigid template that may impede final formation as with current graft materials. The bioactive additives of the implantable devices can also be engineered to provide a chemical reaction known to selectively stimulate osteoblast proliferation or other cellular phenotypes.

The bioactive materials have a relatively small diameter, and in particular, a diameter in the range of about 500 nanometers to about 2,000 microns, or about 0.1 to 50 microns, or a diameter in the range of about 0.1 to about 100 microns. In one embodiment, the diameter can be less than about 10 nanometers, and in another embodiment, the diameter can be about 5 nanometers. In some embodiments, the diameter can be in the range of about 0.5 to about 30 microns. In other embodiments, the diameter can fall within the range of between about 2 to about 10 microns. In still another embodiment, the diameter can fall within the range of between about 3 to about 4 microns.

In some embodiments, further additives can be randomly dispersed throughout the bioactive particles, such as those previously described and including bioactive particles, antimicrobial fibers, particulate medicines, trace elements or metals such as copper, which is a highly angiogenic metal, strontium, magnesium, zinc, etc. mineralogical calcium sources, and the like. Further, the bioactive materials may also be coated with organic acids (such as formic acid, hyaluronic acid, or the like), mineralogical calcium sources (such as tricalcium phosphate, hydroxyapatite, calcium carbonate, calcium hydroxide, calcium sulfate, or the like), antimicrobials, antivirals, vitamins, x-ray opacifiers, or other such materials.

In a normal tissue repair process, at the initial phase a fibrin clot is made that provides a fibrous architecture for cells to adhere. This is the cornerstone of all connective tissue healing. It is this fibrous architecture that allows for direct cell attachment and connectivity between cells. Ultimately, the goal is to stimulate cell proliferation and osteogenesis in the early healing phase and then allow for physiologic remodeling to take place. Since the desired end product is a living tissue and not an inert scaffold, the primary objective is to stimulate as much living bone as possible by enhancing the natural fiber network involved in initiation and osteogenesis.

The materials of the present disclosure may be both osteoconductive as well as osteostimulative to further enhance and accelerate bone induction. Further, the dynamic nature of the bioactive components of the present disclosure allows for natural initiation and stimulation of bone formation rather than placing a non-biologic template that may impede final formation as with current graft materials. The materials disclosed herein can also be engineered to provide a chemical reaction known to selectively stimulate osteoblast proliferation or other cellular phenotypes.

The present disclosure provides bioactive materials and implants formed from these materials. These bioactive materials provide the necessary biocompatibility, structure and clinical handling for optimal healing at the tissue site. In addition, these bioactive materials provide an improved mechanism of action for bone regrowth, by allowing the new tissue formation to be achieved through a physiologic process rather than merely from templating. Further, these artificial bioactive materials can be manufactured as required to possess varying levels of porosity, such as nano, micro, meso, and macro porosity. The bioactive materials can be selectively composed and structured to have differential or staged resorption capacity, while being easily molded or shaped into clinically relevant shapes as needed for different surgical and anatomical applications. Additionally, these bioactive materials may have variable degrees of porosity, differential bioresorbability, compression resistance and radiopacity. These bioactive materials also possess antimicrobial properties as well as allows for drug delivery. The materials can also be easily handled in clinical settings.

The implantable devices may be load bearing, or non-load bearing devices. The devices may be partially or fully resorbable. The devices may be applicable for use in all areas of the body, such as for example without limitation, the spine, shoulder, wrist, hip, knee, ankle, or sternum, as well as other joints like finger and toe joints. Other anatomical regions that can utilize this technology include the dental region and the maxillofacial region, such as the jaw or cheeks, as well as the skull region. The devices may be shaped and sized to accommodate the specific anatomical region to which it is being applied.

In some embodiments, the composite implantable devices of the present disclosure comprise a first, interbody fusion cage component and a second, bioactive component incorporated into the fusion cage component. The two components work in synchrony to produce an overall improved bone fusion device. The spinal fusion devices may be one of a PLIF, TLIF, CIF, ALIF, LLIF or OLIF cage, or a vertebral replacement device. The devices may also be wedge shaped. The spinal fusion devices may be inserted into a patient's intervertebral disc space for restoring disc height to the spinal column.

The implantable devices of the present disclosure may be used for certain components of cortical vertebral spaces or interbody devices, such as spacers, rings, bone dowels, and the like.

The implantable devices of the present disclosure may be incorporated into devices suitable for implantation in the cervical or lumbar regions of a patient's spine. These devices may include artificial discs designed for disc replacement, interbody cages that serve primarily as space holders between two vertebrae, vertebral plates and the like.

In other embodiments, the implantable device of the present disclosure may be used in a variety of orthopedic procedures involving bone repair and restoration. For example, the implantable device may be formed into joints, rods, pins, suture fasteners, anchors, repair devices, rivets, staples, tacks, orthopedic screws, interference screws, bone sleeves, and a number of other shapes that are known in the art. For example, the bioactive composites of the present disclosure may be incorporated into a cortical bone sleeve, or may be inserted into a broken bone as a screw, pin or the like.

The implantable devices of the present disclosure may also be shaped into other orthopedic devices including, but not limited to, sheets, bone plates and bone plating systems, bone scaffolds, bone graft substitutes, bone dowels and other devices useful in fixing bone damaged by trauma or surgery.

The implantable devices of the present disclosure may be shaped into various implants used for total hip arthroplasty, fracture fixation or total knee arthroplasty. For example, the materials of the present disclosure may be used for the stem, the spherical head, a femoral hip dowel and/or the cup assembly of a hip implant. Alternatively, the devices may be used as a receptable sleeve to accommodate a ball joint implant or prosthesis.

The devices of the present disclosure may be used for the bulk restoration or repair of certain defects in bone or oncology defects, such as cortico-cancellous defect fillers, bone graft substitutes or the like.

In other embodiments, the devices of the present disclosure may be used for dental implants, craniomaxillofacial implants, mandibular implants, zygomatic reconstruction and the like. Dental implants, for example, may be placed into the maxilla or mandible to form a structural and functional connection between the living bone.

The implantable devices of the present disclosure may be constructed to provide a connected pathway that directs the growth of bone. For instance, channels or porous networks may be provided to allow communication between the rigid structural framework and the bioactive component additive to allow true interconnectivity and synchrony during the fusion process. This can be accomplished by providing a rigid structural framework that is at least partially porous, or one that may be porous after implantation, when the bioactive material is resorbed and leaves behind a porous opening within the rigid structural framework.

The bioactive component that is the additive to the main body of the implant should be one that will act in synergy with the main body to allow the implantable devices to support cell proliferation and new tissue growth over time. The bioactive additive should provide the necessary porosity and pore size distribution to allow proper vascularization, optimized cell attachment, migration, proliferation, and differentiation. In one embodiment, the bioactive component comprises bioactive glass.

The bioactive material additives of the present disclosure may be in the form of frit, fibers, pellets, powder, microspheres, granules or other particles that are mixed with frit, fibers, pellets, powder, granules, microspheres or other particles of the polymer to form a bioactive composite. By the term granules, what is meant is at least one fragment or more of material having a non-rod shaped form, such as a rounded, spherical, globular, or irregular body. The bioactive additive may be provided in a materially pure form. The bioactive material may comprise fused particles, morsels or porous granules, such as porograns, which are highly porous granular spherical particles that typically have larger surface areas available for cellular activity. The bioactive composite may be further processed and/or combined with the main body into a shaped implantable device having the appropriate properties to withstand the forces required of the implant.

The bioactive material additives may include silica-based materials, boron-based materials and/or strontium-based materials or any combinations thereof. The bioactive material may be glass-based, ceramic-based, a hybrid glass ceramic material that is partially amorphous and partially crystalized or any combination thereof. For example, the bioactive material additive may include one or more of sol gel derived bioactive glass, melt derived bioactive glass, silica based bioactive glass, silica free bioactive glass such phosphate based bioactive glass, crystallized bioactive glass (either partially or wholly), and bioactive glass containing trace elements or metals such as copper, zinc, strontium, magnesium, zinc, fluoride, mineralogical calcium sources, and the like. Examples of sol gel derived bioactive glass include S70C30 characterized by the general implant of 70 mol % SiO₂, 30 mol % CaO. Examples of melt derived bioactive glass include 45S5 characterized by the general implant of 46.1 mol % SiO₂, 26.9 mol % CaO, 24.4 mol % Na₂O and 2.5 mol % P₂O₅, S53P4, and 58S characterized by the general implant of 60 mol % SiO₂, 36 mol % CaO and 4 mol % P₂O₅. Another suitable bioactive glass may also be 13-93 bioactive glass.

The bioactive glass may also comprise at least one alkali metal, for example, lithium, sodium, potassium, rubidium, cesium, francium, or combinations thereof. In once such embodiment, the bioactive glass comprises regions of combeite crystallite morphology. Such bioactive glass is referred to herein as “combeite glass-ceramic”.

The boron-containing bioactive material may include borate or other boron-containing materials, such as a combination of boron and strontium.

In certain embodiments, the bioactive material may be coated with certain materials. The bioactive material may be silanated or silanized, such that its surface is substantially covered with organofunctional alkosilane molecules. Suitable organofunctional alkosilane molecules includes, but are not limited to, aminosilanes, glycidoxysilanes, mercaptosilanes and the like. Silanization of the bioactive materials increases its hydrophobicity and may create a chemical bond that increases it mechanical strength. In addition, silianization of the bioactive material increases the overall Ph of the material, thereby slowing down degradation and potentially controlling the resorption rate.

Further, the bioactive materials may be formed having varying diameters and/or cross-sectional shapes, and may even be drawn as hollow tubes. Additionally, the fibers may be meshed, woven, intertangled and the like for provision into a wide variety of shapes.

The bioactive additives may be engineered with fibers having varying resorption rates. The resorption rate of a fiber is determined or controlled by its material composition and by its diameter. The material composition may result in a slow reacting vs. faster reacting product. Similarly, smaller diameter fibers can resorb faster than larger diameter fibers of the same implant. Also, the overall porosity of the material can affect resorption rate. Materials possessing a higher porosity mean there is less material for cells to remove. Conversely, materials possessing a lower porosity mean cells have to do more work, and resorption is slower. A combination of different fibers may be included in the component in order to achieve the desired result.

In certain embodiments, different areas of the implantable device may have different concentrations of bioactive glass. This provides for different levels of bioactivity and/or resorption throughout the implantable device. In certain embodiments, the outer surface or exterior of the polymer may have greater concentrations of bioactive additive than the interior such that the outer surface reacts with bone tissue more quickly than the interior.

In certain embodiments, for example, one or more of the outer layer(s) of the polymer component may have a concentration of about 0-100 percent bioactive additive and 0-100 percent polymer; whereas the inner layers may have a concentration of about 0-100 percent bioactive additive and about 0-100 percent polymer. In one such example, the outer layer comprises about 40% to 100% bioactive glass and about 0% to about 60% polymer and the interior comprises about 5% to about 40% bioactive material additive and about 60% to about 95% polymer. In another example the outer surface may comprise about 75% to about 100% bioactive material additive and about 0% to about 25% polymer and the inner portions comprises about 5% to about 25% bioactive material additive and about 75% to about 95% polymer.

In certain embodiments, the ratio of weight of the bioactive glass particles to the boron-containing bioactive particles in the device is selected to provide staged resorption of these particles within the body. In an exemplary embodiment, the ratio of weight is about 0 to 1. In another embodiment, the ratio of a particle size of the bioactive glass to a particle size of the boron-containing bioactive particles is selected to provide staged resorption within the body. In an exemplary embodiment, the ratio of particle sizes is about 1 to 0. In other embodiments, both the ratio of weight and the ratio of particle sizes is selected in combination to provide staged resorption within the body.

Similar to the bioactive fibers, the inclusion of bioactive granules can be accomplished using particulates having a wide range of sizes or configurations to include roughened surfaces, very large surface areas, and the like. For example, granules may be tailored to include interior lumens with perforations to permit exposure of the surface of the granule's interior. Such granules would be more quickly absorbed, allowing a tailored implant characterized by differential resorbability. The perforated or porous granules could be characterized by uniform diameters or uniform perforation sizes, for example. The porosity provided by the granules may be viewed as a secondary range of porosity accorded the devices. By varying the size, transverse diameter, surface texture, and configurations of the bioactive glass fibers and granules, if included, the manufacturer has the ability to provide a bioactive glass additive with selectively variable characteristics that can greatly affect the function of the implant before and after it is implanted in a patient. The nano and micro sized pores provide superb fluid soak and hold capacity, which enhances the bioactivity, and accordingly, the repair process.

As previously discussed, the ideal implantable device must possess a combination of features that act in synergy to allow the bioactive agent to support the biological activity of tissue growth and mechanism of action as time progresses. It is known that porosities and pore size distribution play a critical role in the clinical success of implantable fusion devices. More specifically, the devices need to include an appropriate pore size distribution to provide optimized cell attachment, migration, proliferation and differentiation, and to allow flow transport of nutrients and metabolic waste. In addition, in a porous structure the amount and size of the pores, which collectively form the pore size gradient, will be directly related to the mechanical integrity of the material as well as affect its resorption rate. Having a stratified porosity gradient will provide a more complex resorption profile for the devices, and engineering the devices with a suitable pore size gradient will avoid a resorption rate that is too fast or too slow.

Desirably, pore size distribution includes a range of porosities that includes macro, meso, micro and nano pores. A nanopore is intended to represent a pore having a diameter below about 1 micron and as small as 100 nanometers or smaller, a micropore is intended to represent a pore having a diameter between about 1 to 10 microns, a mesopore is intended to represent a pore having a diameter between about 10 to 100 microns, and a macropore is intended to represent a pore having a diameter greater than about 100 microns and as large as 1 mm or even larger. Accordingly, the bioactive glass additive may be provided with variable degrees of porosity, and is preferably ultraporous. In one embodiment, the material may have a range of porosities including macro, meso, micro and nano pores. The resultant engineered implantable device may also include the same range of porosities, which could be provided as a porous network of matrices within the rigid structural framework. Accordingly, porosity may be provided inherently by the actual bioactive glass material itself, or by the porosity of the rigid structural framework.

The bioactive glass and/or the boron-containing material may be provided in a materially pure form. Additionally, the bioactive glass may be mixed with a carrier for better clinical handling, such as to make a resin, putty or foam material. A pliable material in the form of a resin or putty may be provided by mixing the bioactive glass with a flowable or viscous carrier. A foam material may be provided by embedding the bioactive glass in a porous matrix such as collagen (either human or animal derived) or porous polymer matrix. One of the advantages of a foam material is that the porous carrier can also act as a site for attaching cells and growth factors, and may lead to a better managed healing.

In certain embodiments, the implantable device may include a bioactive composite cage that includes a resin, putty or foam material therein.

The carrier material may be porous and may help contribute to healing. For example, the carrier material may have the appropriate porosity to create a capillary effect to bring in cells and/or nutrients to the implantation site. The carrier material may also possess the chemistry to create osmotic or swelling pressure to bring in nutrients to the site and resorb quickly in the process. For instance, the carrier material may be a polyethylene glycol (PEG) which has a high affinity to water.

In some cases, a dry matrix of bioactive glass and/or boron-containing granules and microspheres can be mixed with polymers such as collagen, polyethylene glycol, poly lactic acid, polylactic-glycolic acid, polycaprolactone, polypropylene-polyalkylene oxide co-polymers; with polysaccharides such as carboxymethy cellulose, hydroxypropyl methyl cellulose, with glycosaminoglycan such as hyaluronic acid, chondroitin sulfate, chitosan, N-acetyl-D-glucosamine, or with alginates such as sodium alginate. The dry matrix when hydrated and mixed forms a putty that can be used as mixed, or the product can be loaded into a syringe with a threaded plunger and delivered percutaneously. Alternately, the product can be mixed inside the syringe and delivered percutaneously to form the implantable device in situ.

Equally as important as the material composition and diameter is the pore size distribution of the open porosity and in particular the surface area of the open porosity. The present bone graft components provide not only an improved pore size distribution over other bone graft materials, but a higher surface area for the open pores. The larger surface area of the open porosity of the present implants drives faster resorption by body fluids, allowing the fluid better access to the pores.

Similar to the bioactive glass fibers, the inclusion of bioactive glass granules can be accomplished using particulates having a wide range of sizes or configurations to include roughened surfaces, very large surface areas, and the like. For example, granules may be tailored to include interior lumens with perforations to permit exposure of the surface of the granule's interior. Such granules would be more quickly absorbed, allowing a tailored implant characterized by differential resorbability. The perforated or porous granules could be characterized by uniform diameters or uniform perforation sizes, for example. The porosity provided by the granules may be viewed as a secondary range of porosity accorded the devices. By varying the size, transverse diameter, surface texture, and configurations of the bioactive glass fibers and granules, if included, the manufacturer has the ability to provide a bioactive glass bone graft material with selectively variable characteristics that can greatly affect the function of the implant before and after it is implanted in a patient. The nano and micro sized pores provide superb fluid soak and hold capacity, which enhances the bioactivity and accordingly the repair process.

Due to the pliability of this fibrous graft material, these same bioactive glass fibers may be formed or shaped into fibrous clusters with relative ease. These clusters can be achieved with a little mechanical agitation of the bioactive glass fibrous material. The resultant fibrous clusters are extremely porous and can easily wick up fluids or other nutrients. Hence, by providing the bioactive glass material in the form of a porous, fibrous cluster, even greater clinical results and better handling can be achieved.

One of the benefits of providing an ultra-porous bioactive glass material in cluster form is that handling of the material can be improved. In one manner of handling the cluster of materials, the clusters may be packaged in a syringe with a carrier, and injected into the fusion cage or directly into the bone defect with ease. Another benefit is the additional structural effect of having a plurality clusters of fibers closely packed together, forming additional macrostructures to the overall scaffold of material. Like a sieve, the openings between individual clusters can be beneficial such as when a filter is desired for various nutrients in blood or bone marrow to concentrate certain desired nutrients at the implant location.

Of course, it is understood that, while the term cluster is used to describe the shape of the materials, such term is not intended to limit the invention to spherical shapes. In fact, the formed cluster shape may comprise any rounded or irregular shape, so long as it is not a rod shape. In the present disclosure, the term fibrous cluster represents a matrix of randomly oriented fibers of a range of sizes and length. Additional granules or particulates of material may be placed randomly inside this matrix to provide additional advantages. A variety of materials and structure can optionally be employed to control the rate of resorption, osteostimulation, osteogenesis, compression resistance, radiopacity, antimicrobial activity, rate of drug elution, and provide optimal clinical handling for a particular application.

The use of fused or hardened fiber clusters may be advantageous in some instances, because the fusing provides relative hardness to the clusters, thereby rendering the hardened clusters mechanically stronger. Their combination with the glass granules further enhances the structural integrity, mechanical strength, and durability of the implant. Because larger sized granules or clusters will tend to have longer resorption time, in previous cases the user had to sacrifice strength for speed. However, it is possible to provide larger sized granules or clusters to achieve mechanical strength, without significantly sacrificing the speed of resorption. To this end, ultra-porous clusters can be utilized as just described for fiber-based and glass-based clusters. Rather than using solid spheres or clusters, the present disclosure provides ultra-porous clusters that have the integrity that overall larger sized clusters provide, along with the porosity that allows for speed in resorption. These ultra-porous clusters will tend to absorb more nutrients, resorb quicker, and lead to much faster healing and remodeling of the defect.

In some embodiments, the fiber clusters may be partially or fully fused or hardened to provide hard clusters. Of course, it is contemplated that a combination of both fused fiber clusters (hard clusters) and unfused or loose fiber clusters (soft clusters) may be used in one application simultaneously. Likewise, combinations of putty, foam, clusters and other formulations of the fibrous graft material may be used in a single application to create an even more sophisticated porosity gradient and ultimately offer a better healing response. In some cases, solid porous granules of the bioactive glass material may also be incorporated into the implant.

Another feature of the engineered implantable devices of the present disclosure is their ability to provide mechanical integrity to support new tissue growth. Not only should the bone graft component provide the appropriate biocompatibility and resorption rate, but the surface area should be maximized to fully support cell proliferation. The engineered component can be selectively composed and structured to have differential or staged resorption capacity, while still being easily molded or shaped into clinically relevant shapes as needed for different surgical and anatomical applications. Additionally, these engineered components may have differential bioresorbability, compression resistance and radiopacity, and can also maximize the content of active ingredient relative to carrier materials such as for example collagen.

The implantable devices formed from these materials are able to sustain tissue growth throughout the healing process. One of the deficiencies of currently available implantable devices is their lack of ability to provide proper mechanical scaffolding while supporting cell proliferation over time. The engineered materials and implants of the present disclosure overcome this problem by providing, among other things, an appropriate combination of porosities (i.e., pore size distribution) and high surface area within a porous bioactive glass infrastructure that serves as an ideal scaffold for tissue growth. More importantly, the range of porosities is distributed throughout the porous bioactive glass infrastructure, which is able to support continued cell proliferation throughout the healing process.

The bioactive particles may have a relatively small diameter, and in particular, a diameter in the range of about 0.1 to about 2,000 microns. In exemplary embodiments, the average diameter of the bioactive glass and/or the boron-based material is between about 0.1 and about 400 microns, or about 50 to about 200 microns.

The average diameter of the PAEK polymer is between about 0.5 to about 4,000 microns. The average diameter may be less than 1,000 microns. In other embodiments, the average diameter of the PAEK polymer is greater than 400 microns. In certain embodiments, the average diameter of the PAEK polymer is between 400 to 1,000 microns. This particle size is ideal for compounding with bioactive and boron-based glasses having a particle, pellet or fiber size of 0.1-200 microns.

In some embodiments, further additives can be randomly dispersed throughout the fibers, such as those previously described and including bioactive glass granules, antimicrobial fibers, particulate medicines, trace elements or metals such as copper, which is a highly angiogenic metal, strontium, magnesium, zinc, etc. mineralogical calcium sources, and the like. Further, the bioactive glass fibers may also be coated with organic acids (such as formic acid, hyaluronic acid, or the like), mineralogical calcium sources (such as tricalcium phosphate, hydroxyapatite, calcium carbonate, calcium hydroxide, calcium sulfate, or the like), antimicrobials, antivirals, vitamins, x-ray opacifiers, or other such materials.

The composite devices may be engineered with fibers having varying resorption rates. The resorption rate of a fiber is determined or controlled by, among other things, its material composition and by its diameter. The material composition may result in a slow reacting vs. faster reacting product. Similarly, smaller diameter fibers can resorb faster than larger diameter fibers. Also, the overall porosity of the material can affect resorption rate. Materials possessing a higher porosity mean there is less material for cells to remove. Conversely, materials possessing a lower porosity mean cells have to do more work, and resorption is slower. Accordingly, the composite device may contain fibers that have the appropriate material composition as well as diameter for optimal performance. A combination of different fibers may be included in the construct in order to achieve the desired result. For instance, the implant may comprise a composite of two or more fibers of a different material, where the mean diameter of the fibers of each of the materials could be the same or different.

Another manner of further enhancing the bioactive additive of the present disclosure is to provide an additional layer or coating of polymer over the material in its individual fiber form. For example, biocompatible, bioabsorbable polymer or film-forming agents such as polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), PEG, PLGA, polyesters and the like are suitable materials for coating or binding the fibrous bioactive glass additive. The resultant product is strong, carveable, and compressible, and may still absorb blood. Other suitable materials also include artificial polymers selected from poly(anhydrides), poly(hydroxy acids), polyesters, poly(orthoesters), polycarbonates, poly(propylene fumerates), poly(caprolactones), polyamides, polyamino acids, polyacetals, polylactides, polyglycolides, polysulfones, poly(dioxanones), polyhydroxybutyrates, polyhydroxyvalyrates, poly(vinyl pyrrolidones), biodegradable polycyanoacrylates, biodegradable polyurethanes, polysaccharides, tyrosine-based polymers, poly(methyl vinyl ether), poly(maleic anhydride), poly(glyconates), polyphosphazines, poly(esteramides), polyketals, poly(orthocarbonates), poly(maleic acid), poly(alkylene oxalates), poly(alkylene succinates), poly(pyrrole), poly(aniline), poly(thiophene), polystyrene, non-biodegradable polyurethanes, polyureas, poly(ethylene vinyl acetate), polypropylene, polymethacrylate, polyethylene, poly(ethylene oxide), and co-polymers, adducts, and mixtures thereof. The material may be partially or fully water soluble.

The bioactive glass may be manufactured by electrospinning, or by laser spinning for uniformity. For example, where the material is desired in a fibrous form, laser spinning would produce fibers of uniform diameters. Further, the bioactive glass fibers may be formed having varying diameters and/or cross-sectional shapes, and may even be drawn as hollow tubes. Additionally, the fibers may be meshed, woven, intertangled and the like for provision into a wide variety of shapes.

Bioactive materials of the present disclosure may be prepared using electrospinning techniques. Electrospinning uses an electrical charge to draw very fine (typically on the micro or nano scale) fibers from a liquid or a slurry. When a sufficiently high voltage is applied to a liquid droplet, the body of the liquid becomes charged. The electrostatic repulsion in the droplet would counteract the surface tension and the droplet is stretched. When the repulsion force exceeds the surface tension, a stream of liquid erupts from the surface. This point of eruption is known as a Taylor cone. If molecular cohesion of the liquid is sufficiently high, the stream does not breakup and a charged liquid jet is formed. As the jet dries in flight, the mode of current flow changes from ohmic to convective as the charge migrates to the surface of the fiber. The jet is then elongated by a whipping process caused by electrostatic repulsion initiated at small bends in the fiber, until it is finally deposited on a grounded collector. The elongation and thinning of the fiber resulting from this bending instability leads to the formation of uniform fibers with nanometer-scale diameters.

While the voltage is normally applied to the solution or slurry in a regular electrospinning process, according to embodiments of the present disclosure, the voltage is applied to the collector, not to the polymer solution (or slurry), and, therefore, the polymer solution is grounded. The polymer solution or slurry is sprayed into fibers while applying the voltage in this manner, and the fibers are entangled to form a three-dimensional structure.

The biocompatible polymeric coating may be heat wrapped or heat shrunk around the underlying fibrous bioactive glass additive. In addition, the polymer component may be a mixture of polymer and other components. For example, it is contemplated that the polymer component can comprise 100% of a particular polymer, such as for instance, PLA. However, a mixture of 50% PLA and 50% PEG may also be utilized. Likewise, the polymer component may be formed of a polymer-BAG composition. In this case, the polymer component could comprise 50% polymer with the remaining 50% comprising BAG granules or fibers, for instance. Of course, it is understood that the percentage of an individual component may vary as so desired, and the percentages provided herein are merely exemplary for purposes of conveying the concept.

The embodiments of the present disclosure are not limited, however, to fibers alone. In other embodiments, the additive may be bioactive granules or powder. These granules may be uniform or non-uniform in diameter, and may comprise a mixture of differently sized diameters of granules. In addition, the granules may be formed of the same type of bioactive glass material, or a mixture of different materials selected from the group of suitable materials previously mentioned. The granules may be solid or porous, and in some cases a mixture of both solid and porous granules may be used. Regardless, the engineered implant comprising the granular foundation should still provide the desired pore size distribution, which includes a range of porosities that includes macro, meso, micro and nano pores.

Like the fibers, at least a portion of the surface of the bioactive composite may be coated with a polymeric coating. The coating may be solid or porous. In other embodiments, the coating could comprise collagen or hydroxyapatite (HA). For instance, the coating could be a solid collagen or a perforated collagen. Added surface features including fibers, granules, particulates, and the like can be included in the coating to provide an exterior with bioactive anchorage points to attract cellular activity and improve adhesion of the implant in situ.

In some embodiments, at least some or all of the engineered composite implantable device may be coated with a glass, glass-ceramic, or ceramic coating. The coating may be solid or porous. In one embodiment, the coating may be a bioactive glass such as 45S5 or S53P4. In still further embodiments, the implants may comprise a multi-layered composite of varying or alternating materials. For example, in one case a bioactive glass fiber or granule may be encased in a polymer as described above, and then further encased in a bioactive glass. This additional bioactive glass layer could be the same as, or different, than the underlying bioactive glass. The resultant construct would therefore have varying resorption rates as dictated by the different layers of materials.

In addition, the incorporation of biological agents such as glycosaminoglycans and/or growth factors may also provide cell signals. These factors may be synthetic, recombinant, or allogenic, and can include, for example, stem cells, demineralized bone matrix (DBM), as well as other known cell signaling agents.

In some embodiments, the engineered composite implantable devices may be also osteoconductive and/or osteostimulatory. By varying the diameter and chemical composition of the components used in the embodiments, the engineered implants may have differential activation (i.e., resorbability), which may facilitate advanced functions like drug delivery of such drugs as antibiotics, as an example. One manner of providing osteostimulative properties is to incorporate bone marrow into the bioactive glass fiber additive. The incorporation of the marrow would produce an osteostimulative implantable device that accelerates cell proliferation.

In other embodiments, the engineered composite implantable device may also include trace elements or metals such as copper, zinc, strontium, magnesium, zinc, fluoride, mineralogical calcium sources, and the like. These trace elements provide selective benefits to the engineered structural and functioning implants of the present disclosure. For example, the addition of these trace elements like strontium may increase x-ray opacity, while the addition of copper provides particularly effective angiogenic characteristics to the implant. The materials may also be coated with organic acids (such as formic acid, hyaluronic acid, or the like), mineralogical calcium sources (such as tricalcium phosphate, hydroxyapatite, calcium sulfate, calcium carbonate, calcium hydroxide, or the like), antimicrobials, antivirals, vitamins, x-ray opacifiers, or other such materials. These bioactive glass additives may also possess antimicrobial properties as well as allow for drug delivery. For example, sodium or silver may be added to provide antimicrobial features. In one embodiment, a layer or coating of silver may be provided around the implantable device to provide an immediate antimicrobial benefit over an extensive surface area of the implant. Other suitable metals that could be added include gold, platinum, indium, rhodium, and palladium. These metals may be in the form of nanoparticles that can resorb over time.

Additionally, biological agents may be added to the implantable device. These biological agents may comprise bone morphogenic protein (BMP), a peptide, a bone growth factor such as platelet derived growth factor (PDGF), vascular endothelial growth factor (VEGF), insulin derived growth factor (IDGF), a keratinocyte derived growth factor (KDGF), or a fibroblast derived growth factor (FDGF), stem cells, bone marrow, and platelet rich plasma (PRP), to name a few. Other medicines may be incorporated into the devices as well, such as in granular or fiber form. In some cases, the bioactive glass additive can serve as a carrier for the biological agent, such as BMP or a drug, for example.

The implantable device may be a custom device that is designed for an individual patient's specific anatomy. The size and shape of the implantable device may be based, for example, on patient CT scans, MRIs or other images of the patient's anatomy, In certain embodiments, these images may be used to form a customized device through additive manufacturing techniques, such as selective layer melting (SLM), selective laser sintering (SLS), E-beam or 3D printing of metal, metal alloy or polymer, and fused deposition modeling (FDM). In other embodiments, the images may be used to create molds for forming the customized device.

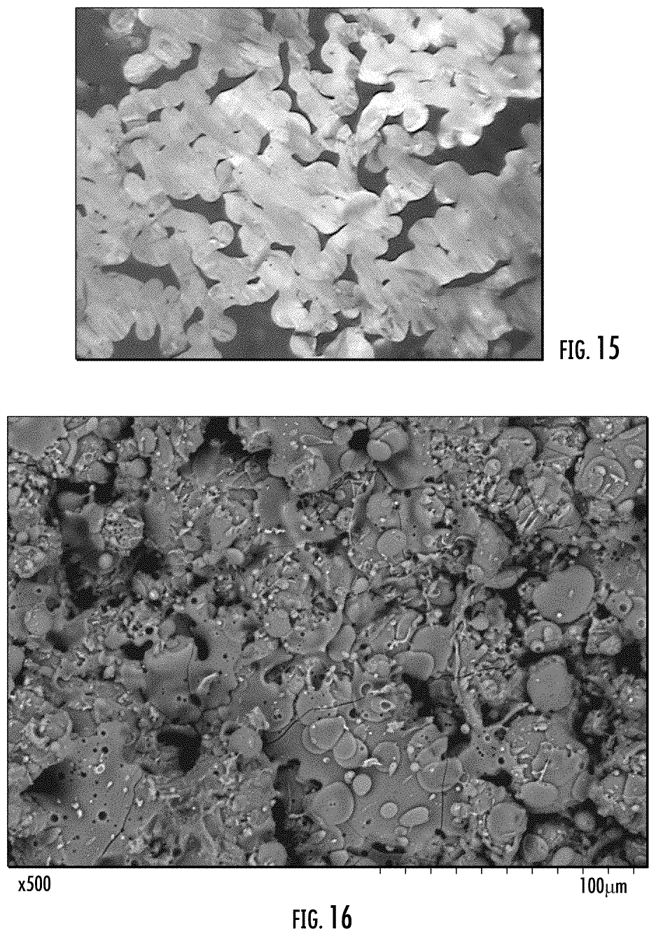

FIG. 1 illustrates one example of an implantable device 100 according to the present disclosure. As shown, device 100 includes a main body 102 substantially surrounded by a bioactive component 104 that may include any of the bioactive materials described above. In this embodiment, the bioactive component 104 substantially covers the entire outer surface of main body 102 to enhance cellular activity and promote bone fusion and/or regrowth around this surface. This will maximize the potential to chemically and physiologically bond tissue to relatively non-reactive materials like PEEK and improve upon purely mechanical bonding that hydroxyapatite or titanium sprayed surfaces offer. FIG. 15 illustrates an example of a bioactive glass that has been applied to the surface of a titanium alloy. The surface is completely covered with bioactive glass, and offers a porous surface microstructure that is ideal for tissue adhesion and enhancing the tissue-implant interface.

Main body 102 may comprise any suitable material, such as a polymer, metal, ceramic or a combination of the above. Bioactive component 104 preferably comprises a polymer, such as PAEK, combined with a bioactive additive. The bioactive additive may comprise any of the bioactive materials described herein.

FIG. 16 illustrates pores for cellular attachment in direct contact with a material that is known to chemically react in-vivo and form a strong calcium phosphate surface that bone and soft tissue can attach to, but actually integrate and eventually form a functional tissue interface. This interface will not only promote tissue healing, but bioactive glass has been known for decades to have anti-infective properties useful in combating bacteria and fungi that may come in contact with the load bearing implant. This coating adds a layer of protection against biofilm caused by colonizing bacteria and is expected to enhance the life of medical implants by reducing the likelihood of infection at the implant site.

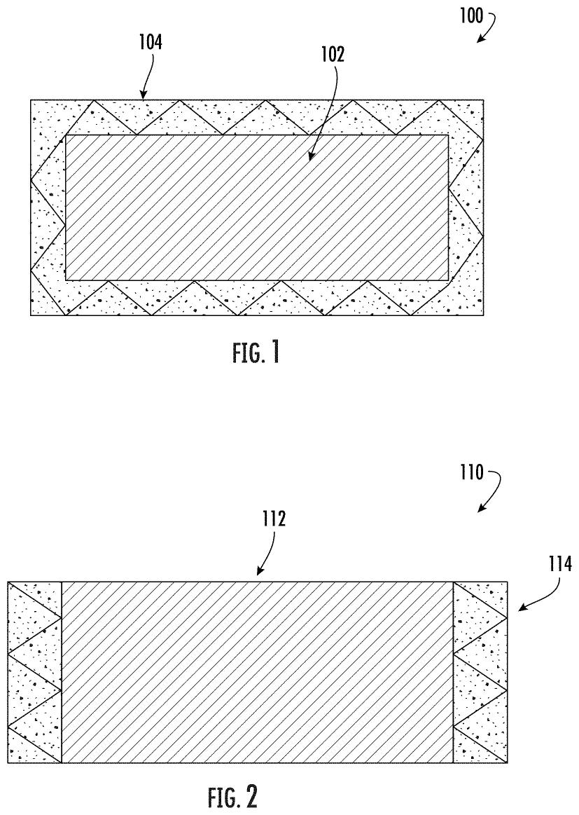

FIG. 2 illustrates another example of an implantable device 110 according to the present disclosure. Device 110 includes a main body 112 and a bioactive component 114 that is present on at least some portions of the outer surface of main body 112. In this embodiment, the bioactive component 114 is preferentially disposed on either end of main body 112 to enhance cellular activity on these ends. Of course, it will be recognized that other configurations are possible. For example, bioactive component 114 may be disposed on only end of main body 112, and/or it may be disposed on one or more of the bottom and top surfaces of main body 112. Alternatively, bioactive component 114 may be disposed at discrete locations around the outer surface of main body 111, e.g., in linear or non-linear strips, random or non-random locations around the surface, and the like.

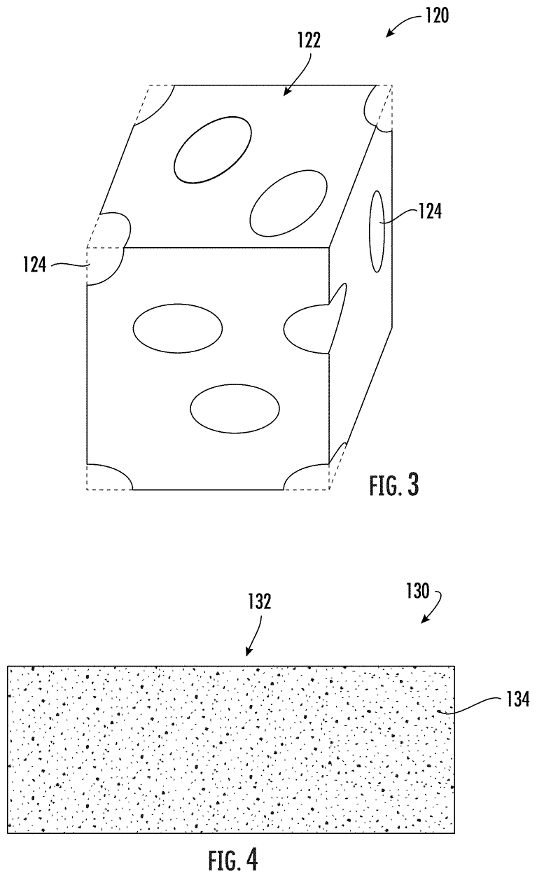

FIG. 3 illustrates a porous implantable device 120 having a main body 122 and a number of pores 124 interspersed throughout main body 122. A bioactive component (not shown) has been incorporated into main body 122 in or around pores 124. The bioactive component interacts with the cellular tissue, allowing for bone regrowth into pores 124, as discussed above. This embodiment uses bioactive materials to leave behind a network of pores and channels that will be used by infiltrating tissues to essentially grow through the load bearing implant. This tissue infiltration throughout the implant will impart some fraction of the load onto living tissue which is imperative to combat stress shielding. It will also reduce the volume of implant material and allow more room for regenerated tissue over time. Some embodiments only have surface features to promote more of a mechanical bond while other embodiments strive to promote tissue to penetrate completely through the implant.

FIG. 4 illustrates yet another example of an implantable device 130 having a main body 132 and a bioactive component 134 that has been interspersed throughout main body 132. In this embodiment, main body 132 may comprise the polymer component (e.g., a PAEK material). Alternatively, main body 132 may comprise a different material, such a different polymer, a ceramic or metal and bioactive component 134 will comprise both a PAEK material and the bioactive materials discussed herein. The bioactive component 134 may be mixed with the main body 132 in particle form, and then processed in one of the methods discussed below.

The overall implantable device 130 may be substantially homogenous, i.e., the bioactive component 134 and the main body 130 are mixed together such that the overall implant 130 has substantially the same properties throughout. Alternatively, the bioactive component 134 and the main body 130 may be non-homogenous such that the bioactive component 134 is interspersed through main body 130.

FIG. 5 illustrates an embodiment of an implantable device 140 that includes one or more layers. In the example shown, a bioactive layer 144 is sandwiched between two other layers 142, 146 of non-bioactive material, such as metal, ceramic and/or polymer materials. Of course, other configurations are possible. For example, the layer of non-bioactive material may be sandwiched between layers of bioactive materials. In addition, the device 140 may include 2 layers, or 4 or more layers of bioactive and non-bioactive materials alternating throughout the device.

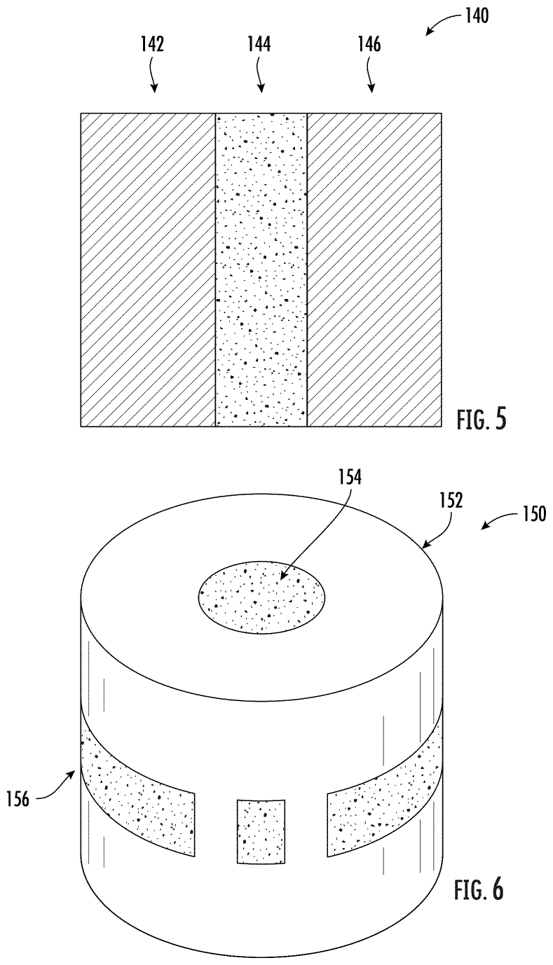

FIG. 6 illustrates a cage component 150 of an implantable device that may be used, for example, between two adjacent vertebral bodies in a fusion procedure. As shown, cage component 150 includes a main body 152 that may include open cavities which may then be partially or fully filled with bioactive materials 154, 156, such as those described above. If desired, allograft material may be included. The packed metallic cage and bone graft material construct may be put into a collage matrix or slurry with the addition of a binder to create a multi-composition device.

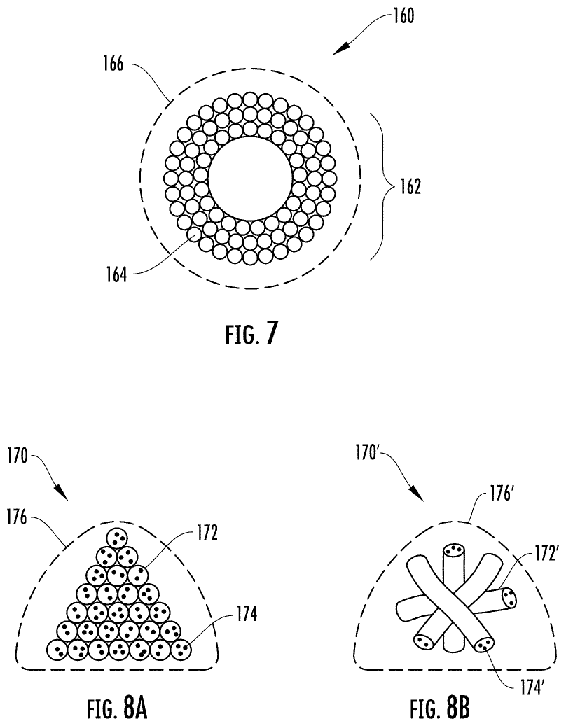

The bioactive component of the composite implantable devices may be fibrous in nature, and comprise bioactive glass fibers. These fibers may be specifically aligned for directionality. In one example, as shown in FIG. 7, the composite implantable device 160 may comprise bundles 162 of individual fibers 164, with the fibers 164 being unidirectional within a particular bundle 162. A coating 166 may optionally be provided around the bundles 162. The bundles 162 may be arranged in a particular pattern, such as in a cylinder, as illustrated.

Directionally aligned bioactive components add a connectivity that is unique from other types of devices as the bioactive components pull liquid from one end to the other. This connectivity will enhance and direct the growth of tissues and ultimately improve the mechanical bond between the implant and surrounding tissues. The pores present in the directional fiber assemblage of the present disclosure will promote the migration of hard and soft tissue in the spaces between the fibers. In addition, the fibers may be configured to promote the circulation of liquids through capillary action that occurs between the fibers. This constant movement of fluids will enhance tissue growth as oxygen and nutrients are brought into the implant and metabolic waste products are removed. This capillary action will continue indefinitely until the fibers are filled with new tissue and the forces between body fluids and the pore volume are eliminated.

In other exemplary embodiments, the individual bundles may be selectively aligned, so as to provide an overall effect of purposeful directionality. For example, FIG. 8A shows a composite implantable device 170 in which a plurality of bundles 172 of individual fibers 174 are uniformly aligned, and which may optionally include a coating 176 surrounding the bundles 172. FIG. 8B shows a composite implantable device 170′ in which a plurality of bundles 172′ of individual fibers 174′ are randomly aligned to provide multidirectionality. The plurality of fibers 174, 174′ within each bundle 172, 172′ allow for robust cellular growth, while also controlling the directionality of the growth. An optional coating 176, 176′ may be provided for each device 170, 170′.

The fiber bundles shown in FIGS. 7, 8A and 8B may be incorporated into a composite implantable device. In such a design, the fiber bundles may be at least partially, if not fully, contained within a main body of the implantable device and selectively aligned relative to the device to provide directionality of cell growth through the device. The fiber bunders may be uniformly aligned with each other, or they may be aligned in different directions relative to each other. For example, the fiber bundles can extend along one or more axes of the implantable device to provide cell growth along those axes. In another example, the fiber bundles may be randomly oriented relative to each other, but selectively aligned relative to the implantable device. In all of these examples, the main body of the implantable device may include a polymer with bioactive materials incorporated throughout the polymer according to any of the embodiments disclosed herein. Additional examples of implantable devices incorporating fiber bundles can be found in commonly-assigned, co-pending U.S. patent application Ser. No. 16/151,774, filed Oct. 4, 2018, the complete disclosure of which is incorporated herein by reference in its entirely for all purposes as if copied and pasted herein.

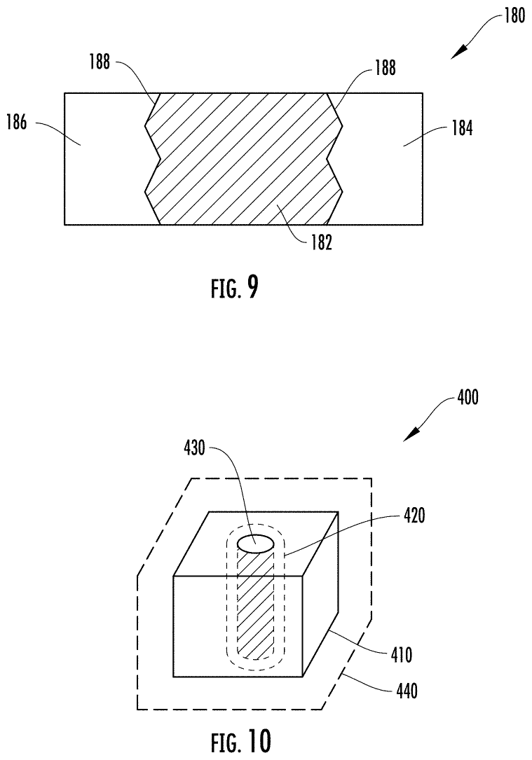

In still another embodiment shown in FIG. 9, the composite implantable device 180 may comprise multiple interlocking components. For instance, the polymer component(s) and bioactive material component(s) may include shaped connection surfaces like threads, fins, a dovetail, tongue and groove, shark's tooth, and other similar structural features that allow for individual components to interlock onto one another. In addition, the bioactive material component may comprise oriented fibers, morsels, or a combination of both. As shown, a bioactive component main body 182 may have an interlocking end that allows caps 184, 186 to lock on at these interlocking junctions 188.

In another exemplary embodiment shown in FIG. 10, the cage component 410 of the composite implantable device 400 may be a PEEK (polyetheretherketone) cage, with PEEK being a temperature sensitive material. In its simplest form, the cage 410 may have a bone graft containment chamber 420 for receiving the bone graft component 430. As illustrated, in one embodiment, the containment chamber 420 may be filled with a plug 430 formed of bioactive glass. The plug 430 may comprise fibers, morsels, or any combination thereof. The fibers may also be aligned or not aligned, as described earlier. In other embodiments, this containment chamber 420 may be tapered to allow ease of packing material therein. The cage may have a wedge shape to facilitate its insertion. The cage may be pre-filled with the bone graft component and be encapsulated. For instance, the entire cage plus graft component may be coated or covered with a skin 440 of material such as those previously mentioned above. The coating or skin may or may not be porous. Further, surface features may be provided on the coating or skin.

Suitable filler material may include BAG fibers, BAG morsels, microspheres containing drugs or other active agents, or a collagen slurry, for instance. If desired, allograft material may be included. The allograft material may include bone chips, stem-cell preserved bone chips, or human-derived collagen. These package materials may also be pre-treated or wetted, such as with a solution like water, saline, blood, bone marrow aspirate, or other suitable fluids. Bone cement may also be used.

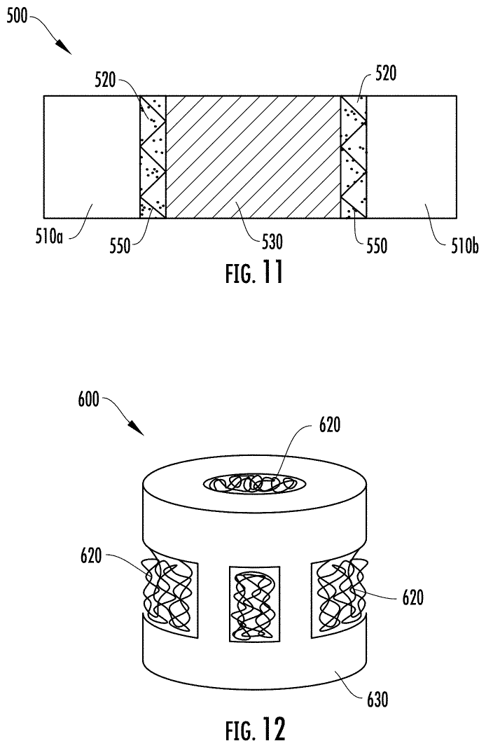

Referring now to FIG. 11, the internal cavity of the composite implantable device 500 may include flexible features to allow bending, in order to accept the graft plug or component, but may flex back to its original shape in order to keep the graft plug in place. For instance, BAG fibers may be used pre-packed with the cage component such that the fibers act as a liner or gasket and allow the BAG plug to be secured to the PEEK cage component(s) with a degree of flexibility until fully locked into place.

As illustrated, a composite implantable device 500 has a main body comprising a bioactive glass component or plug 530, similar to the one shown in FIG. 10. The ends of the plug 530 may have an interlocking junction 550 to cooperate with end caps 510a, 510b which may be formed of PEEK, for example. The interlocking junction 550 may include threads as an example. Surrounding the threads may be BAG fibers 520, as shown.

As mentioned, the cage component of the composite implantable devices may be temperature resistant or non-temperature sensitive. Such cage components may be formed of a metal, for instance. As illustrated in FIG. 12, in another exemplary embodiment, the metallic cage 630 of the composite implantable device 600 may include open cavities 620 which may then be partially or fully filled with bone graft material 620. The bone graft material 620 may be bioactive glass in the form of fibers or morsels, as described above. If desired, allograft material may be included. The packed metallic cage and bone graft material construct 600 may be put into a collage matrix or slurry with the addition of a binder to create a multi-composition device.

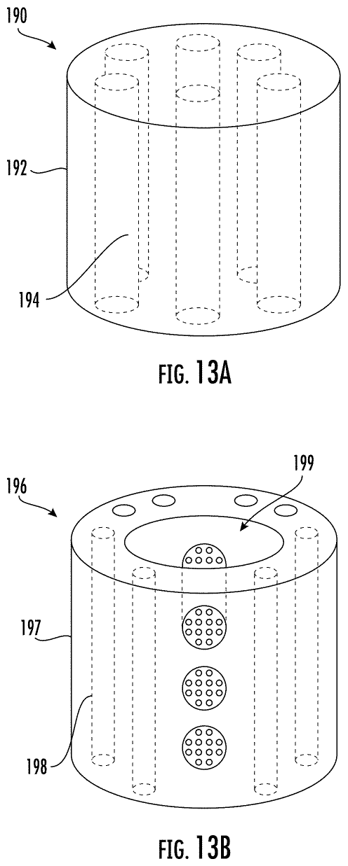

FIGS. 13A and 13B illustrate another embodiment of an implantable device that includes an assemblage of directionally aligned bioactive glass fibers that connect one side of the implant with another. Directionally aligned porosity adds a connectivity that is unique from other types of porosity in that the pores pull liquid from one end to the other. This connectivity will enhance and direct the growth of tissues and ultimately improve the mechanical bond between the implant and surrounding tissues.

FIG. 13A illustrates one such embodiment of an implantable device 190 that includes a main body 192 and one or more directionally aligned bioactive components 194. In this embodiment, bioactive components 194 extend from the bottom surface to the top surface of main body 192 and are aligned substantially in this direction to connect one side of the implant with the other.

FIG. 13B illustrates another example of an implantable device 196 with directionally aligned bioactive components. As shown, device 196 includes a main body 197 having a central channel 199 and one or more bioactive components 198 forming elongate tubes that extending from a top surface of the device 196 to the bottom surface and arrayed around central channel 199. Device 196 may further include one or more bioactive components 198 within central channel 199.

The pores present in the directional fiber assemblage of the present disclosure will promote the migration of hard and soft tissue in the spaces between the fibers. In addition, the fibers may be configured to promote the circulation of liquids through capillary action that occurs between the fibers. This constant movement of fluids will enhance tissue growth as oxygen and nutrients are brought into the implant and metabolic waste products are removed. This capillary action will continue indefinitely until the fibers are filled with new tissue and the forces between body fluids and the pore volume are eliminated.

Porosity in a foam or more spherical shape pulls liquid into the pore, but then there is no driving force to move it along to recycle fluids. Incorporation of this aligned pore network through a load bearing implant will enhance healing and tissue growth in a non-obvious compared to the traditional implants that have a large void present. The use of aligned porosity to not just be a void for tissue to fill, but also orienting the pores to add a dynamic flowing fluid functionality is unique and an improvement over the state of the art in clinical practice.

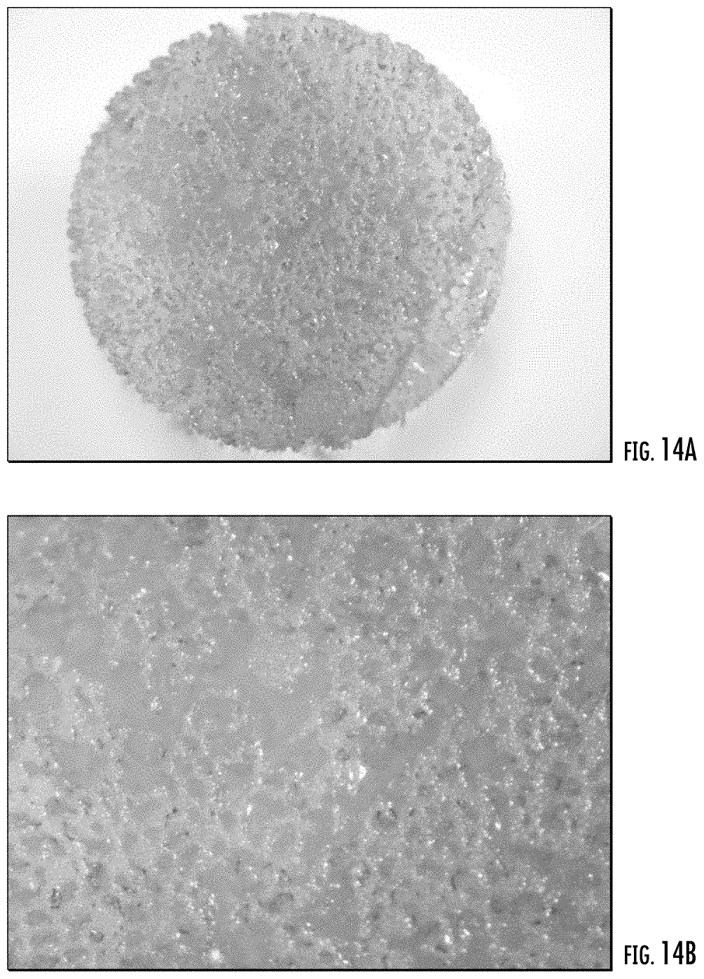

The aligned porosity can also enhance the dispersion of materials such as bone marrow aspirate that are often added to promote healing in load bearing implants prior to implantation. The capillary action of the aligned fibers pulls the cells and body fluids present in the marrow through the assemblage and start the healing process. FIGS. 14A and 14B illustrate a directional fiber assemblage that has been infiltrated with a cell suspension of MLOA-5 bone cells. FIG. 14B is a magnified view, and the dark spots are bone cells that have been stained to better identify them. These cells were pulled through from the other end of the assemblage to illustrate the benefits of aligned fibers.

FIG. 15 illustrates a magnified view of a bioactive load bearing implant with an additional bioactive glass coating covering the entire load bearing implant. This will maximize the potential to chemically and physiologically bond tissue to relatively non-reactive materials like PEEK and improve upon purely mechanical bonding that hydroxyapatite or titanium sprayed surfaces offer. The surface is completely covered with bioactive glass, and offers a porous surface microstructure that is ideal for tissue adhesion and enhancing the tissue-implant interface.

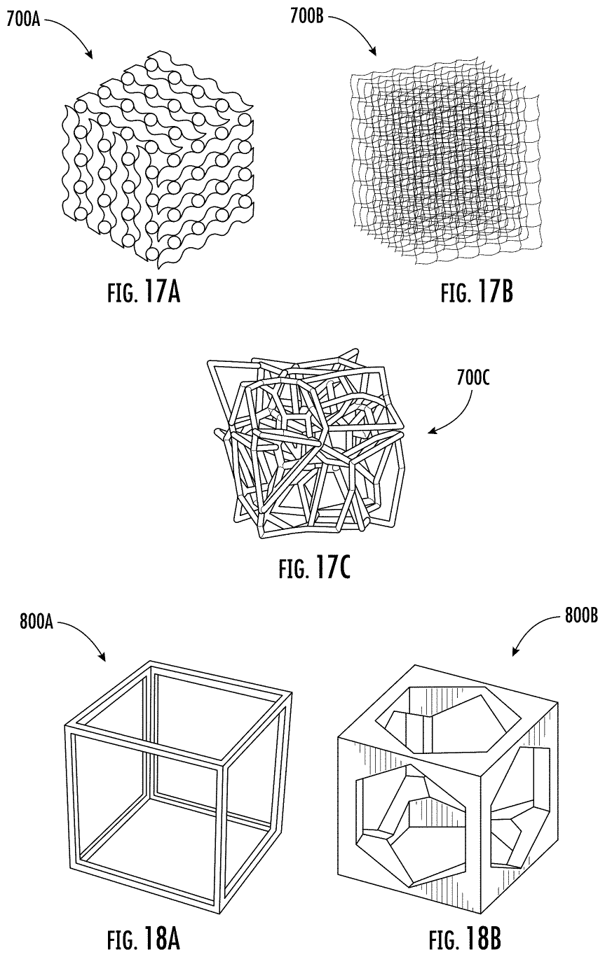

FIGS. 17A to 17C illustrate examples of implantable devices formed form lattice structures 700A, 700B, 700C. Lattices are regular, three-dimensional repeating structure that allow for the creation of porous lattices in, for example, orthopedic implants. As shown in FIGS. 17A, 17B and 17C, these porous lattice structures 700A, 700B and 700C provide room for osseointegration by providing a scaffold to encourage cell on-growth and in-growth into the pore spaces. The empty spaces within the lattice allows for fluids and nutrients to enter the implant, thereby allowing for osteointegration of bone tissue. The scaffold may be formed from metal, ceramic or polymer material and may also include a bioactive component, as described above. Alternatively, the lattice structure itself may be created in-vivo with bioactive or resorbable materials that either dissolve or assimilate into the bone tissue.

In certain embodiments, the lattice structure implants of the present disclosure may be designed to incorporate two separate phases in-vivo. In the first phase, fluids and nutrients are allowed to pass into the empty spaces of the lattice to provide for osteointegration. In the second phase, the actual lattice framework may be formed form completely or partially from resorbable materials (as discussed above) such that the entire structure, or a portion of the structure, dissolves, thereby leaving only bone tissue behind.

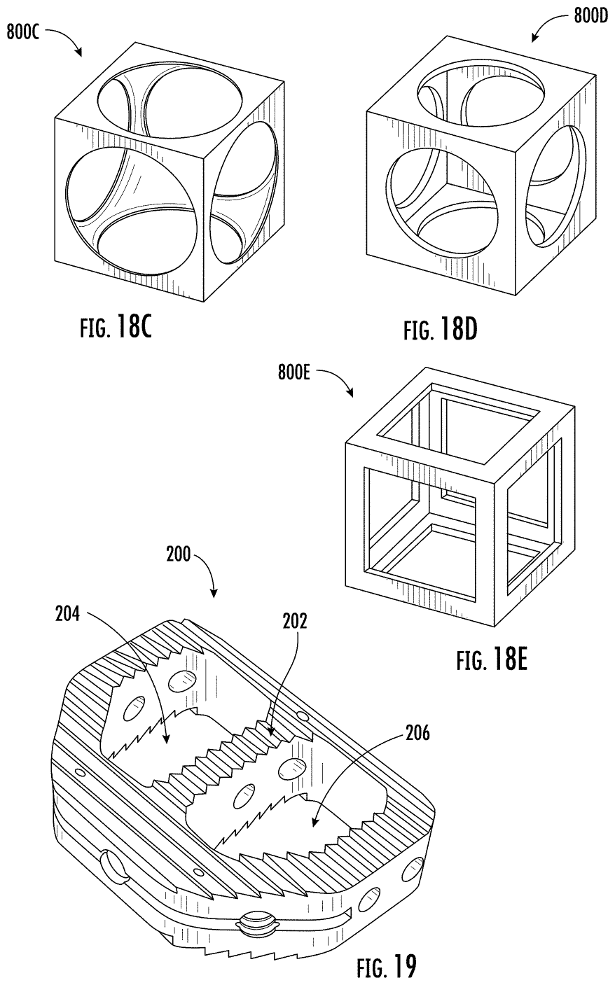

The lattice structures of the present disclosure may include repeating units of geometric structure, or they may be formed with random geometric structures throughout the lattice. FIGS. 18A to 18E illustrate examples of repeating geometric structures 800A, 800B, 800C, 800D, 800E, that may be formed within a lattice-type implant according to the present disclosure. Of course, other repeating structures may be used, such as diamond-shaped, square, trapezoidal, triangular, spherical, cylindrical, and the like.

The bioactive material of the present disclosure may be incorporated into devices suitable for implantation in the cervical or lumbar regions of a patient's spine. These devices may include artificial discs designed for disc replacement, interbody cages that serve primarily as space holders between two vertebrae, vertebral plates and the like. FIG. 19 illustrates various aspects of one embodiment of a cervical implant 200 of the present disclosure. The cervical implant 200 may be formed from the composite bioactive polymeric material of the present disclosure. Implant 200 may vary in size to accommodate differences in the patient's anatomy. The implant 200 is comprises an anterior side a posterior side and a pair of opposing sidewalls. The implant 200 may include an internal wall 202 extending from the anterior side to the posterior side. Internal wall 202 creates two open spaces 204, 206 for the placement of graft material therein. The graft material may comprise allograft material, autograft material, or synthetic materials. The synthetic graft material may comprise a biocompatible, osteoconductive, osteoinductive, or osteogenic material to facilitate the formation of a solid fusion column within the patient's spine.

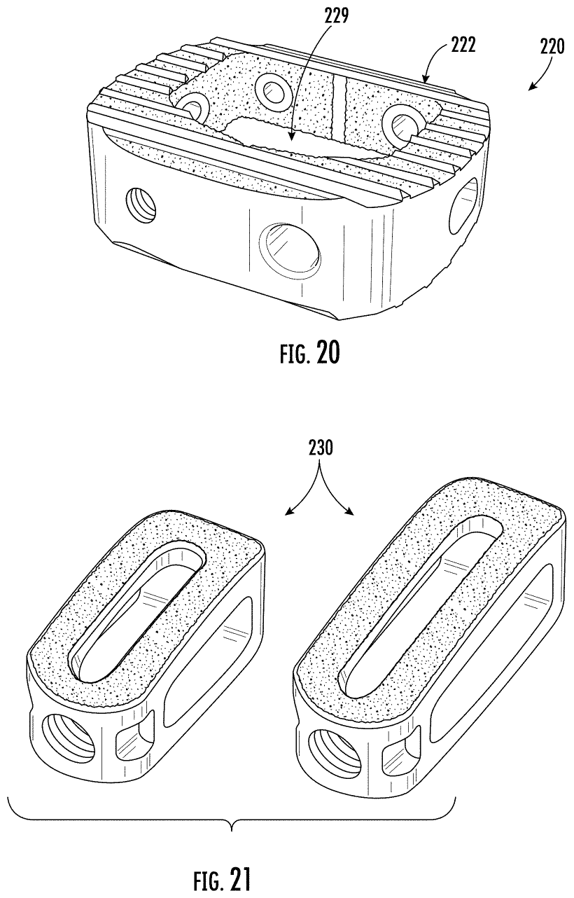

FIG. 20 illustrates another embodiment of a cervical implant 220 of the present disclosure. Cervical implant 220 is similar to implant 200 shown in FIG. 15 except that it includes an outer framework 222 that encloses a single open space 229 for the placement of graft material therein. Cervical implant 220 may be formed from the composite bioactive polymeric material of the present disclosure.

The bioactive material of the present disclosure may also be formed into an implant suitable for lumbar procedures, such as PLIF, TLIF, ALIF, LLIF or OLIF cages, or a vertebral replacement device. These cages may be formed from the composite bioactive polymeric material of the present disclosure. FIG. 21 illustrates an example of implants 230 suitable for PLIF procedures. The PLIF implant may be a variety of different sizes to accommodate differences in the patient's anatomy or the location in the spine. As shown, implant 230 comprises an anterior side, a posterior side, a lateral side and a medial side. Implant 230 also includes a major recess formed in the body creating a longitudinal through-aperture in communication with the top and bottom surfaces. The convergence of these through-apertures forms a cavity inside the implant in which graft material may be placed.

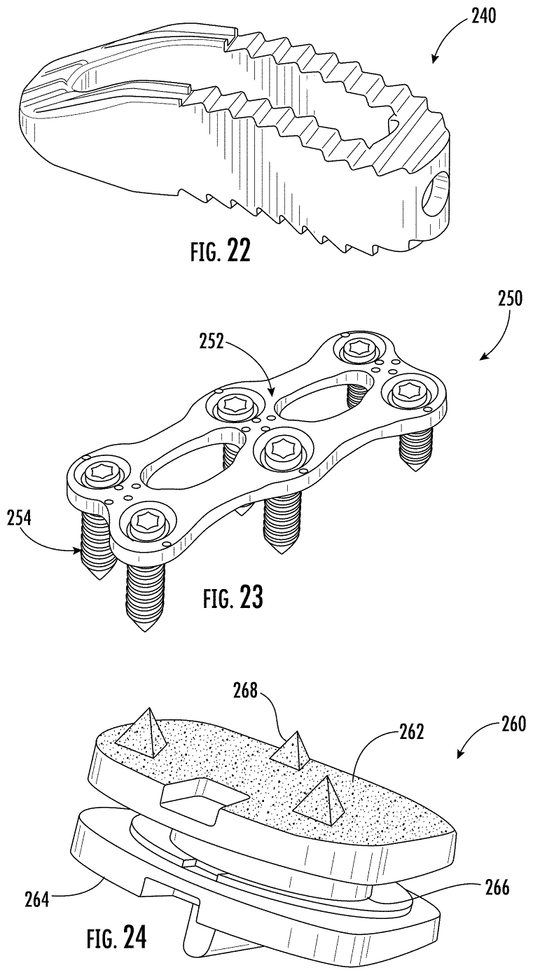

FIG. 22 illustrates an example of an implant 240 suitable for TLIF procedures. The TLIF implant may be a variety of different sizes to accommodate differences in the patient's anatomy or the location in the spine.

FIG. 23 illustrates an embodiment of a cervical plate 250 and fasteners 2\52 that may be used in conjunction with one of the cervical implants described above to enhance neck stability. Cervical plate 250 may be used for a variety of conditions to immobilize, stabilize or align cervical vertebrae. Cervical plate 250 includes an elongated rectangular plate 252 that spans the distance between two adjacent vertebrae. Fasteners 254 may include screws, nails, pins and the like. They are inserted through openings within plate 250 to engage adjoining vertebral bodies. According to the present disclosure, all or a portion of plate 254 and/or fasteners 252 may be formed from a composite material of metal, ceramic or polymer combined with the bioactive materials discussed above.

The bioactive material of the present disclosure may be incorporated into artificial disc implants that are inserted into the lumbar or cervical regions of the spinal column to replace degenerated intervertebral discs. FIG. 24 illustrates an embodiment of an artificial disc implant 260 according to the present disclosure. As shown, disc 260 comprises upper and lower endplates 262, 264 and a movable core 266 therein. Endplates 262, 264 each include anchors 268 for fixating end plates to adjacent vertebral bodies. According to the present disclosure, certain portions of endplates 262, 264 and/or anchors 268 may include a bioactive component incorporated into a metal or ceramic main body to enhance fixation with the adjoining vertebrae.

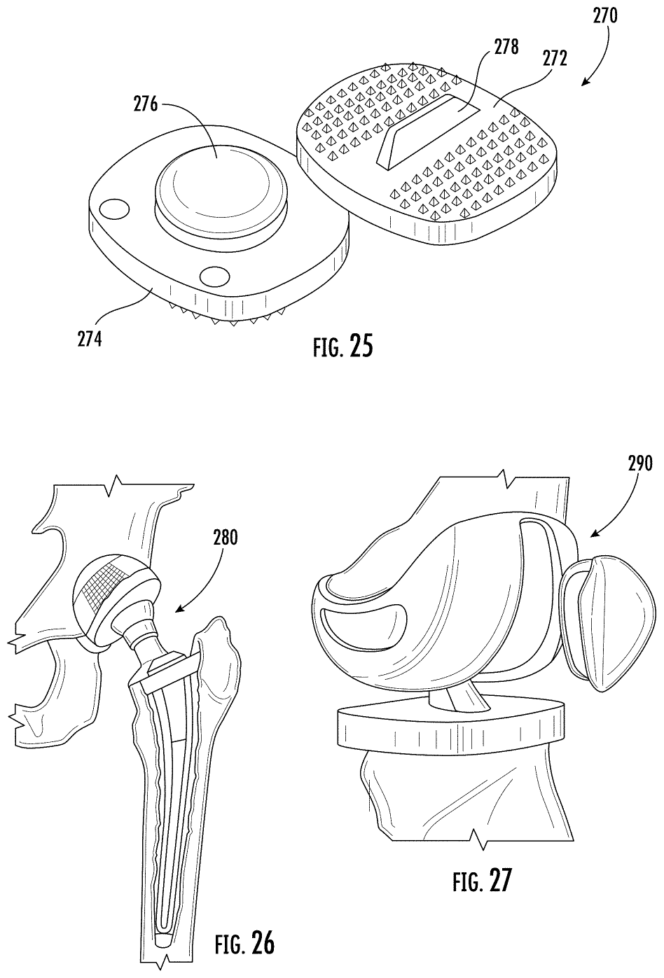

FIG. 25 illustrates another embodiment of an artificial disc 270 that also includes upper and lower endplates 272, 274 and a movable core 276 therein. In this embodiment, each endplate includes one or more keels 278 that extend transversely from the endplates to fixate the endplates into the vertebral bodies. As with the previous embodiment, certain portions of endplates 272, 274 and/or keel(s) 278 may include a bioactive component incorporated therein to enhance fixation with the adjoining vertebrae. For example, all or a portion of the disc implants may be formed from the composite bioactive polymeric material of the present disclosure.

In some aspects of the invention, the composite main body may be used in orthopedic procedures, such as hip or knee arthroplasty. Total hip or knee arthroplasties are surgical procedures in which the hip or knee joint is replaced by a prosthetic. Such joint replacement surgery if generally conducted to relieve arthritis pain or fix severe joint damage. FIG. 26 illustrates one embodiment of a hip implant 280 comprising the bioactive materials of the present disclosure. FIG. 27 illustrates one embodiment of a knee implant 290 comprising the bioactive materials of the present disclosure. The implants may comprise bioactive materials throughout the entire implant, or in parts of the implant. For example, the main body of the implants may be formed from the composite bioactive polymeric material of the present disclosure.

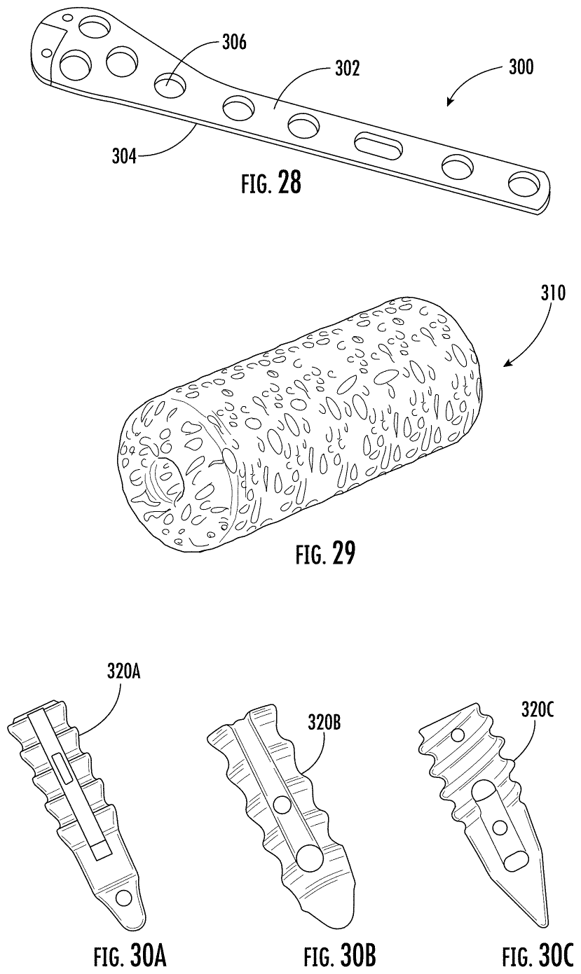

In other aspects of the invention, the composite bioactive framework may be used for bone plates, such as those used to aid in the treatment of different bone fractures and osteotomies. Typically, the bone plate will be specifically designed for a particular anatomical location on the patient. FIG. 28 illustrates one embodiment of a wrist plate 300 that may be, for example, shaped and dimensioned for reduction and compression of fracture(s) in and around the arm and wrist, such as a distal radius or ulna fracture. As shown, bone plates 300 has a plate body with an upper surface 302, a lower, bone contacting surface 304 and medial and lateral side surfaces connecting upper and lower surfaces 302, 304. Bone plate 300 preferably includes one or more bone screw holes 306 configured to receive a plurality of screws (not shown) for fixating plate to the patient's bone. The bioactive component of the present disclosure may be incorporated into the bone screws, or the bone plate. For example, the bone plate or screw may be formed from the composite bioactive polymeric material of the present disclosure.

In other embodiments of the present disclosure, the composite shaped body may be used for certain components of cortical vertebral spaces or interbody devices, such as spacers, rings, bone dowels, and the like. FIG. 29 illustrates one embodiment of a bone dowel 310 that may be used, for example, as a femoral hip dowel that is inserted into a femur requiring restoration. The bone dowel 310 may comprise bioactive materials throughout the entire implant, or in parts of the implant. For example, the bone dowel 310 may be formed from the composite bioactive polymeric material of the present disclosure.

FIGS. 30A to 30C illustrate various embodiments of bone anchors 320A, 320B, 320C that may incorporate the bioactive material of the present disclosure. For example, the bone anchors 320A, 320B, 320C may be formed from the composite bioactive polymeric material of the present disclosure. Bone anchors 320 may comprise screws rods, pins, or other fixation devices that comprise metal or other material with bioactive materials incorporated therein.

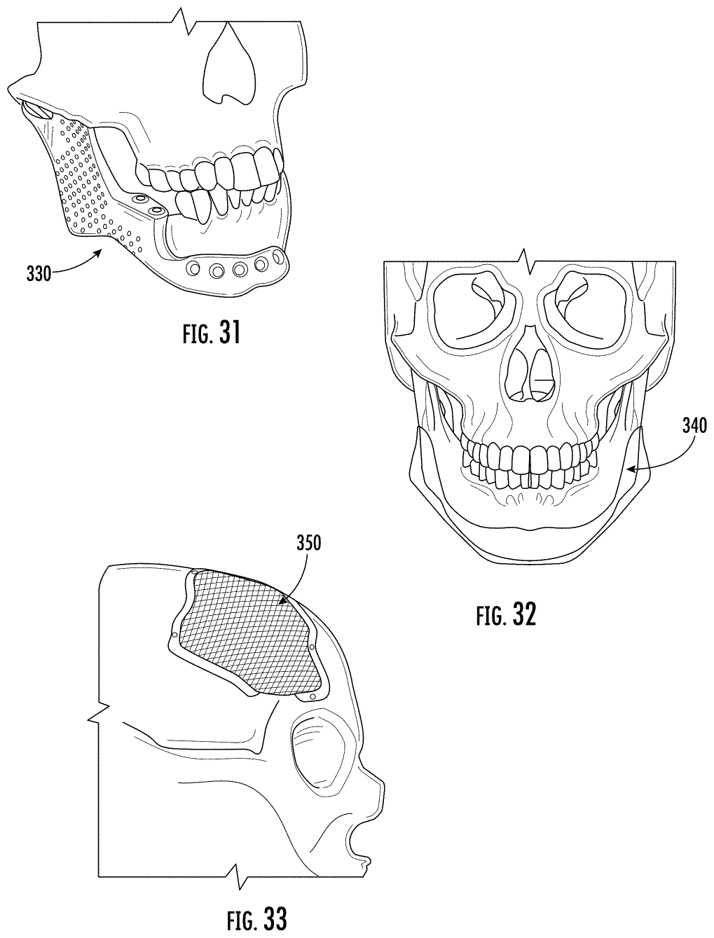

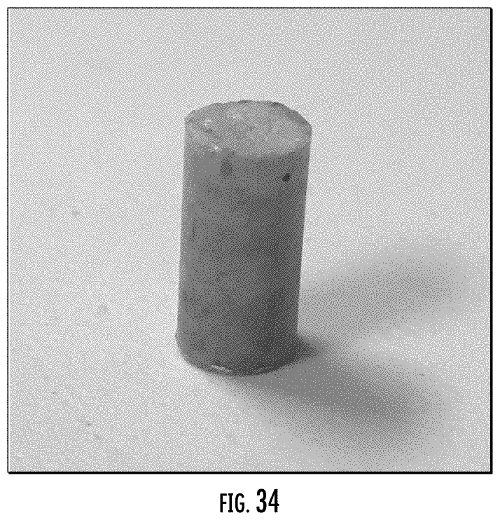

The bioactive composites of the present disclosure may also be formed into the shape of craniomaxillofacial implants or dental implants. These implants may be, for example, placed into the maxilla or mandible to form a structural and functional connection between the living bone. FIGS. 31 and 32 illustrate two different embodiments of jaw implants 330, 340 that may include the bioactive material of the present disclosure. The jaw implants 330, 340 may also be formed from the composite bioactive polymeric material of the present disclosure. FIG. 33 illustrates an embodiment of a cranial implant 350. The cranial implant 350 may include the bioactive material, or may be formed from the composite bioactive polymeric material of the present disclosure.

The present disclosure also provides methods for manufacturing implantable devices that include a polymer, such as PAEK, and a bioactive component, such as bioactive glass and a boron-containing material.

In certain aspects, the implantable device may be formed by an additive manufacturing technique whereby layers of material are formed and then deposited on each other to create the final device. These additive manufacturing techniques may include selective layer melting (SLM), selective laser sintering (SLS), E-beam or 3D printing of metal, metal alloy or polymer, fused deposition modeling (FDM) or combinations.

In these embodiments, the layers of material that are deposited onto each other may each have different concentrations of bioactive glass. This provides for different levels of bioactivity and/or resorption within different portions of the resulting implantable device. In certain embodiments, the outer layers of the polymer may have greater concentrations of bioactive additive than the inner layers such that the outer layers react with bone tissue more quickly than the inner layers. This design creates relatively rapid bioactivity on the outer layers and a longer and slower bioactivity throughout the interior of the device.

In certain embodiments, for example, one or more of the outer layer(s) of the polymer component may have a concentration of about 40-80 percent bioactive additive and 20-60 percent polymer; whereas the inner layers may have a concentration of about 20-60 percent bioactive additive and about 40-80 percent polymer. The relative concentrations may be about 50-75 percent bioactive additive and 25-50 percent polymer in one or more of the outer layer(s) and about 25-50 percent bioactive additive and 50-75 percent polymer in the inner layers.

In other aspects, the methods of the present disclosure mix particles of the polymer and the bioactive materials into a substantially homogenous composite. The particles may be pellets, granules, powder, fibers or the like. The methods of the present disclosure allow for particles of the PAEK and the bioactive component to have different or mis-matched particle sizes prior to mixing them to form the homogenous composite. In addition, the composite device is prepared without the use of a solvent to remove the alkalinity of the bioactive material.

The methods of the present disclosure also allow for the preparing of the bioactive composite without preheating the polymer prior to processing. In addition, the bioactive composite may be prepared in large batches that can be further processed to product shaped implants that have the appropriate mechanical properties to withstand the forces required of spinal, orthopedic, dental or other implants.

In certain embodiments, the resulting product may be subjected to secondary processing that may, for example, include sanding or otherwise roughening the outer surface of the main body after it has been formed. Applicant has discovered that sanding, grit blasting (or otherwise machining) the surface of the bioactive composite device immediately after its formation results in significant bioactivity at substantially the entire surface that is machined. Sanding or otherwise machining the surface may expose particles or micropores within the material that are below the outer surface to allow bone tissue to grow into the main body and/or it may draw the bioactive materials to the surface of the device. In addition, sanding the surface increases the overall surface area of the composite device by creating a rougher surface that has more surface area to interact with bone tissue.

In one embodiment, the process includes mixing particles of a polyaryletherketone (PAEK) polymer and a bioactive additive such as those described above to form a substantially homogenous mixture. The substantially homogenous mixture is then compressed and heated to at least the melting temperature of the particles within the mixture to form a bioactive composite in a shape of the load bearing implantable device.

The polymer and bioactive additive particles may be compression molded in any suitable compression molder designed to apply heat and pressure to force the materials into conform to the shape of a mold cavity. Suitable compression molders for use with the present disclosure include bulk molding compounds (BMC), sheet molding compounds (SMC) and the like.

The method of the present disclosure takes advantage of compression molding techniques such that the polymer and the bioactive material may be inserted into the mold in the form or powder or pellets that have been readily metered by weight. This has the advantage that the bioactive material is mixed with the polymer to produce a substantially homogenous bioactive composite. The polymer particles and the bioactive particles are preferably mixed together without using a solvent to remove the alkalinity of the bioactive material.

In certain embodiments, the particles of PAEK polymer and bioactive additive are in the form of a powder. The bioactive additive may comprise a bioactive glass and a boron-based bioactive material. The boron-based bioactive material may comprise borate. The bioactive glass may comprise Combeite, 45s5 bioactive glass or a combination thereof.

The PAEK polymer particles have an average diameter of less than 100 microns. In some embodiments, the average diameter is about 45 microns to about 65 microns. The borate particles and the 45s5 materials have an average diameter of about 50 microns to about 400 microns. In some embodiments, the average diameter is about 90 microns to about 355 microns.

In one such method, PEEK and bioactive glass powders are mixed together until the mixture appears to be substantially homogenous. The powders may be mixed with any suitable method known to the art, i.e., hand, ball mill or the like. A suitable mold is then placed on the center of an aluminum foil, which is placed onto a metal sheet. The mold cavities are filled with the powder mixture, and the metal sheet and mold are placed into a compression molding machine. The mixture is heated and compressed until the powders reach at least their melting temperature such that they melt together in the mold cavity.

After heating and compression, the mold cavities are allowed to cool down and solidify. Typically, the cooled samples shrink, thereby leaving empty space within the mold cavities. Accordingly, this process may be repeated several times until the cooled specimen completely fills the mold cavities.

In another embodiment, a process for forming a load bearing implantable device comprises mixing particles of a polyaryletherketone (PAEK) polymer and a bioactive additive into a screw extruder, rotating the screw extruder and heating the particles of the PAEK polymer and the bioactive additive to at least a melting temperature of the particles to form a homogenous composite in a shape of the load bearing implantable device. The powders may be mixed with any suitable method known to the art, i.e., hand, ball mill or the like. Extrusion devices that can be employed, for example, include single and twin-screw machines, co-rotating or counterrotating, closely intermeshing twin-screw compounders and the like. In one embodiment, the screw extruder may be a twin screw extruder with two meshing screws that are commonly used to plasticize and extrude plastic materials.

In certain embodiments, the PAEK polymer and the bioactive additive are in the form of powder. The bioactive additive may comprise bioactive glass, such as 45S5 or Combeite and/or boron-based material, such as borate. The process includes mixing the powders of the PAEK polymer and the bioactive additive together to form a homogenous mixture and then placing the homogenous mixture into the screw extruder.

In another embodiment, the PAEK polymer is in the form of pellets and the bioactive additive is in the form of powder. The PAEK pellets are first inserted into the screw extruder and rotated and heated until the pellets form into a powder. The bioactive powder is then mixed into the extruder with the PAEK powder to form a homogenous product. This homogenous product is then further rotated and heated to form a bioactive composite that can be shaped into a load bearing implant.

In yet another embodiment, a method for forming a load bearing implantable device includes mixing particles of a polyaryletherketone (PAEK) polymer and a bioactive additive into a screw extruder and rotating the screw extruder to form homogenous composite pellets. The pellets are then compressed and heated within, for example, a compression molder to at least a melting temperature of the pellets (e.g., about 700 degrees Fahrenheit) to form a bioactive composite in a shape of the load bearing implantable device.

In this embodiment, homogenous pellets are formed that can be re-processed and compression molded into the desired shape. This provides a number of advantages over traditional compression molding processes that are subject to variability in homogeneity, variability in bioactive glass distribution, a higher likelihood of structural imperfections, lower yields and small final shapes.

Of course, other combinations of the above methods can be used according to the present disclosure. For example, the particles of PAEK and bioactive composite may be compression molded into a substantially homogenous composite. This composite may then be extruded through, for example, a twin screw extruder, to form the final implant device. Alternatively, the bioactive components may be compression heated into a surface of the polymer.

### WORKING EXAMPLES

The following are examples of composite materials or engineered implantable devices formed from composite bioactive materials described in the present disclosure:

### Example 1: BAG Powder Additive

A composite material, or an implantable device made from a composite material, may be engineered from a composite of polyetheretherketone (PEEK) or polyetherketoneketone (PEKK) with bioactive materials incorporated into the polymer composite. The device may be in the form of an interbody fusion device. The bioactive materials may take the form of microspheres or powders, and may make up approximately 23% of the composite material. The bioactive materials may be encapsulated in the PEKK or PEEK resin.

The implantable device may be formed using additive manufacturing techniques such selective laser sintering (SLS). The bioactive materials may in the form of a powder, and have an average particle size of 80 microns, with a particle size range of 45 to 115 microns.

### Example 2: BAG Fiber Additive

A composite material, or an implantable device made from a composite material, may be engineered from a composite of polyetheretherketone (PEEK) or polyetherketoneketone (PEKK) with bioactive materials incorporated into the polymer composite. The device may be in the form of an interbody fusion device. The bioactive materials may take the form of fibers. The bioactive fibers may be extruded with the PEKK or PEEK resin.

The implantable device may be formed using additive manufacturing techniques such as fused deposition modeling (FDM). The bioactive glass additive may in the form of fibers that are added to extruded polymeric filaments of the PEKK or PEEK in a layer-by-layer deposition process to build the device. The diameters of the fibers may range from 50 microns or less, to diameters from about 50 to 200 microns. The larger sized diameter fibers may be especially suitable for creating interconnecting porous networks or channels, as they resorb and create empty spaces inside the device.

### Example 3

A composite material, or an implantable device made from a composite material, may be engineered from a composite of polyetheretherketone (PEEK) or polyetherketoneketone (PEKK) with bioactive materials. In these examples, the bioactive materials include MoSci borate glass powders and/or MoSci 45S5 glass powders, although it will be recognized that the device may be formed from any of the bioactive materials described herein. The bioactive additive may comprise 100% borate, 100% 45S5 or a mixture of both (i.e., 50/50 or some other percentage). The overall composition of the device is about 80% PEEK, and 20% bioactive additive (i.e., borate and/or glass powders).

FIG. 34 illustrates such a device that has been engineered from Evonik Vestakeep 2000 FP K15 PEEK material with particles sizes of about 55 microns. The bioactive additive comprises MoSci borate glass powders and/or MoSci 45S5 glass powders, each having particle sizes of about 90 to about 355 microns or about 75 to about 125 microns.

This device was manufactured by compression molding powders of the PEEK particles and bioactive materials together. The product may also be subjected to secondary processing consisting sanding or other machining to increase the surface exposure of the bioactive glass.

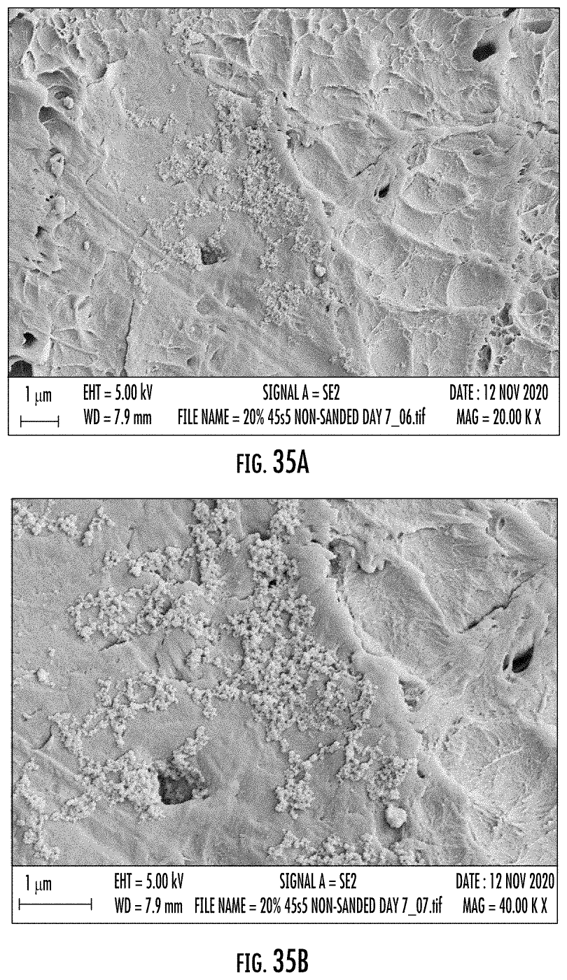

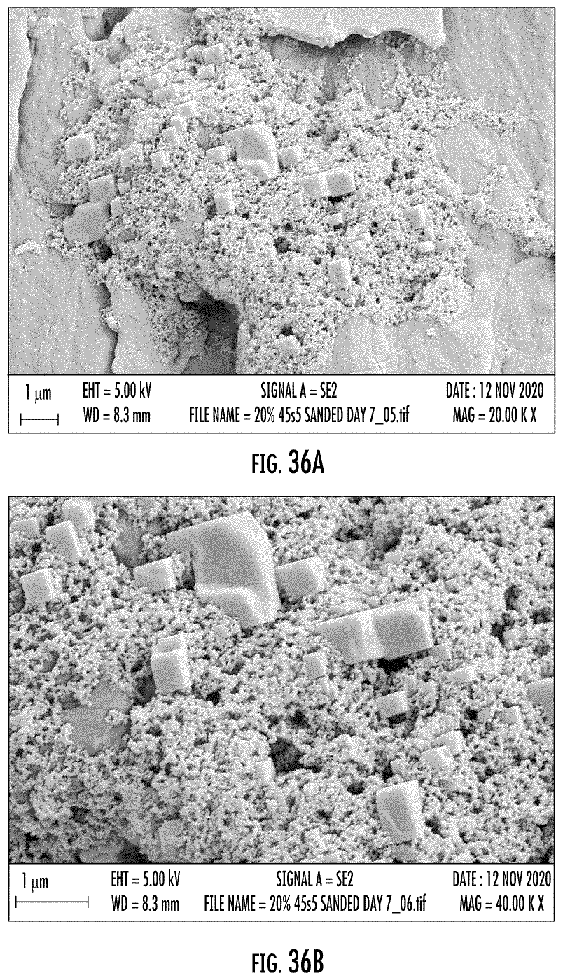

The device in FIG. 34 was sanded in an attempt to expose more borate and 45S5 material at the surface of the device. FIGS. 35A and 35B illustrate two magnified views of a surface of a device comprising 20% 45S5 bioactive glass and 80% PEEK after seven days. These samples were not sanded. FIG. 35A is magnified at 20.00 K X and FIG. 35B is magnified at 40.00 K X. FIGS. 36A and 36B illustrate a device comprising 20% 45S5 bioactive glass and 80% PEEK at seven days after having been sanded, illustrating the bioactivity at the surface of the device.

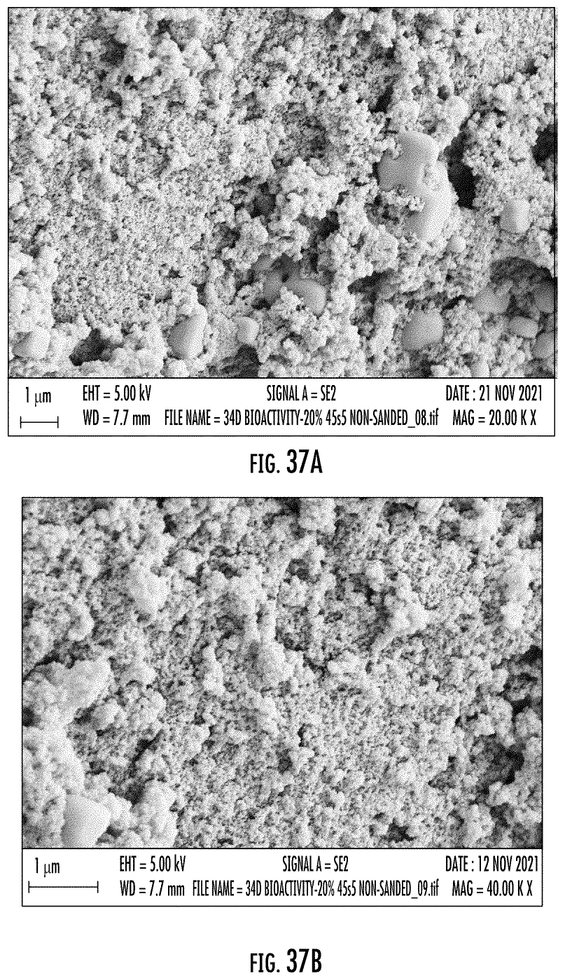

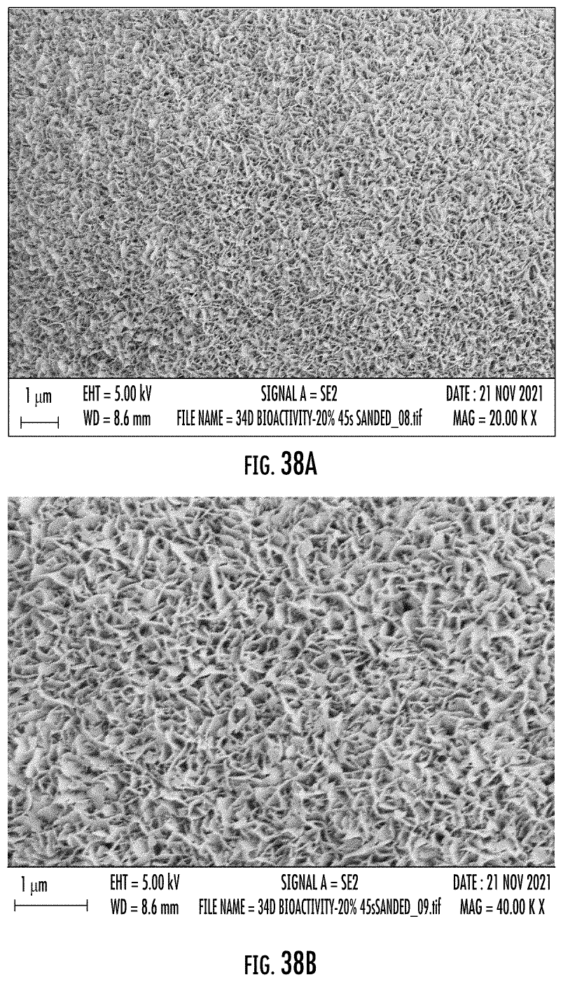

FIGS. 37A and 37B illustrate the bioactivity of the same device of FIGS. 35A and 35B (i.e., non-sanded) after 34 days. FIGS. 38A and 38B illustrate the bioactivity of the same device of FIGS. 36A and 36B (i.e., sanded) after 34 days. As shown, substantially all of the outer surface of the sanded device includes hydroxyapatite, the mineral of the apatite group that is the main inorganic constituent of bone tissue. This clearly shows that almost the entire surface of the sanded device has undergone significant bioactivity.

Applicant has discovered that sanding (or otherwise machining) the surface of the bioactive composite device immediately after its formation results in significant bioactivity around substantially the entire surface of the device. Sanding or otherwise machining the surface draws the bioactive materials to the surface of the device. In addition, sanding the surface increases the overall surface area of the composite device by creating a rougher surface that has more surface area to interact with bone tissue.

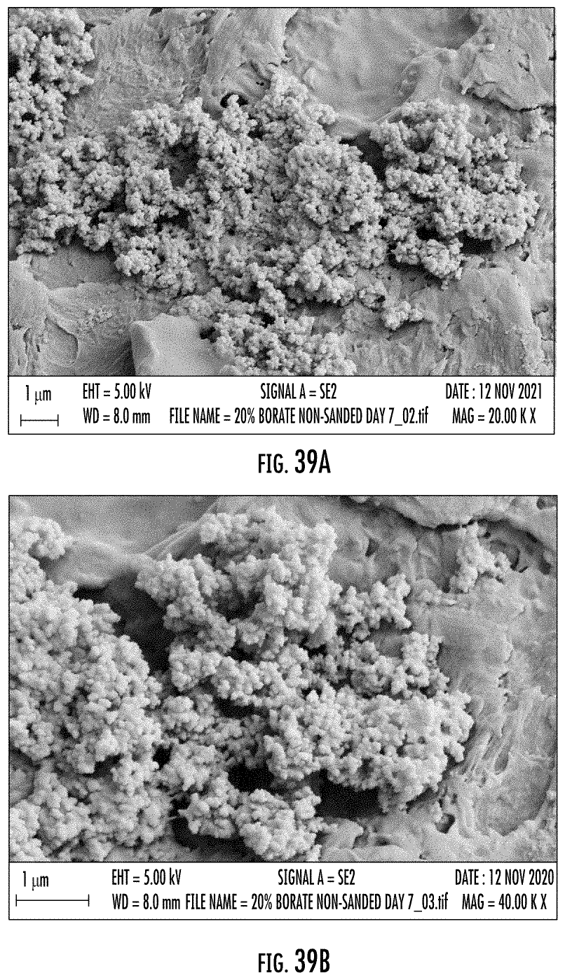

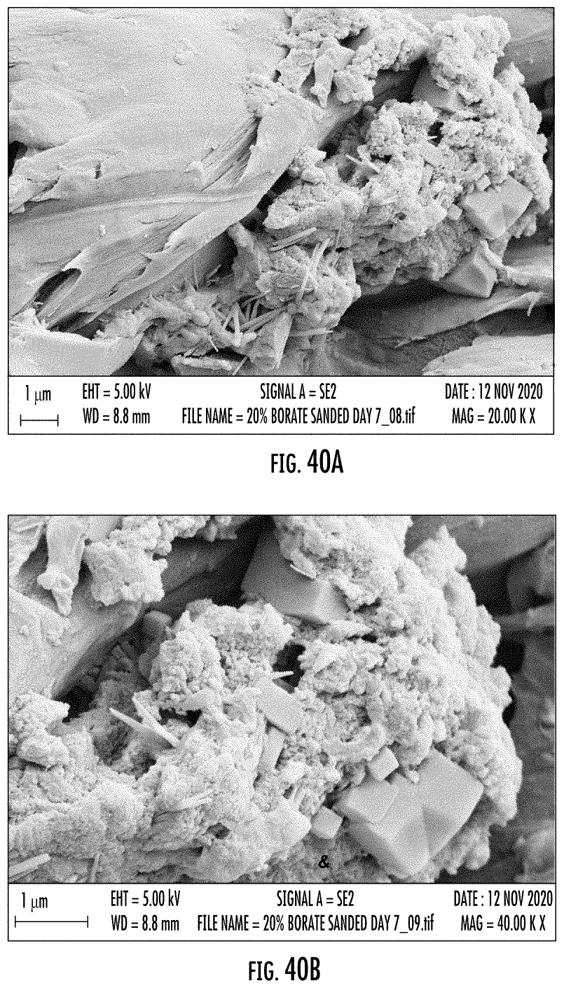

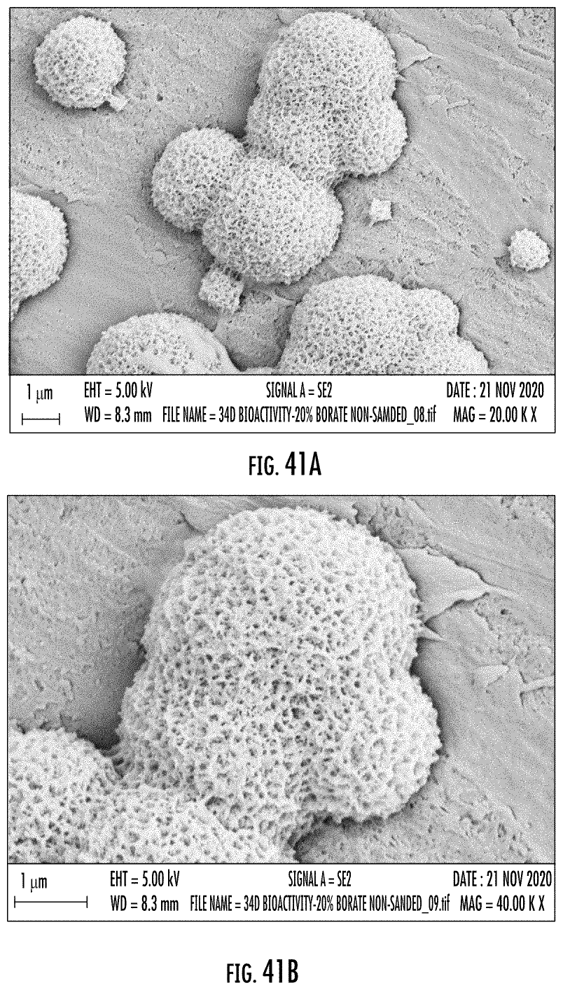

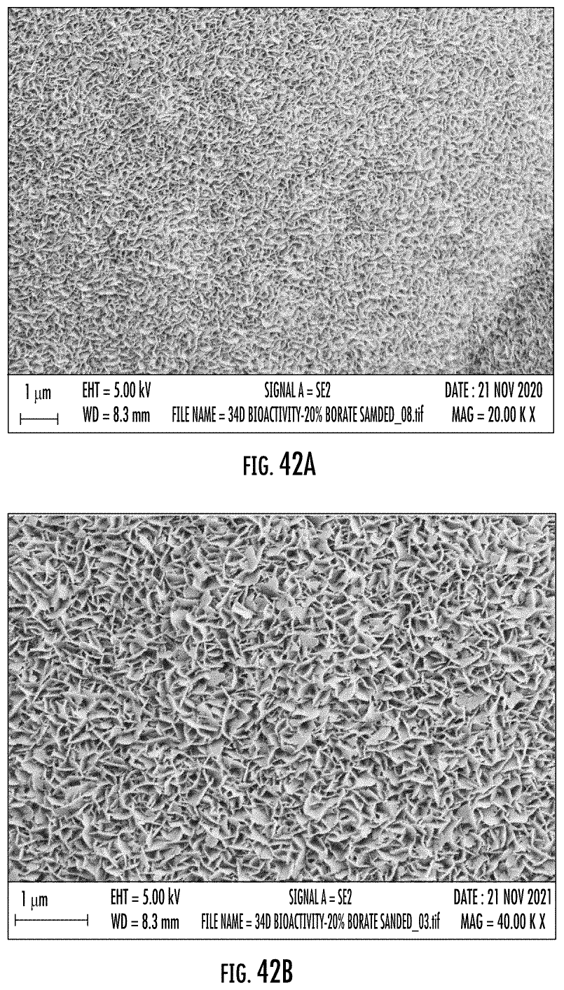

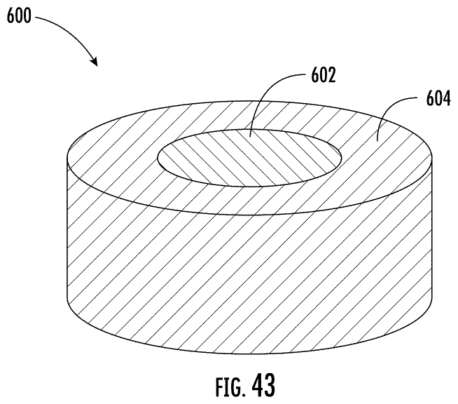

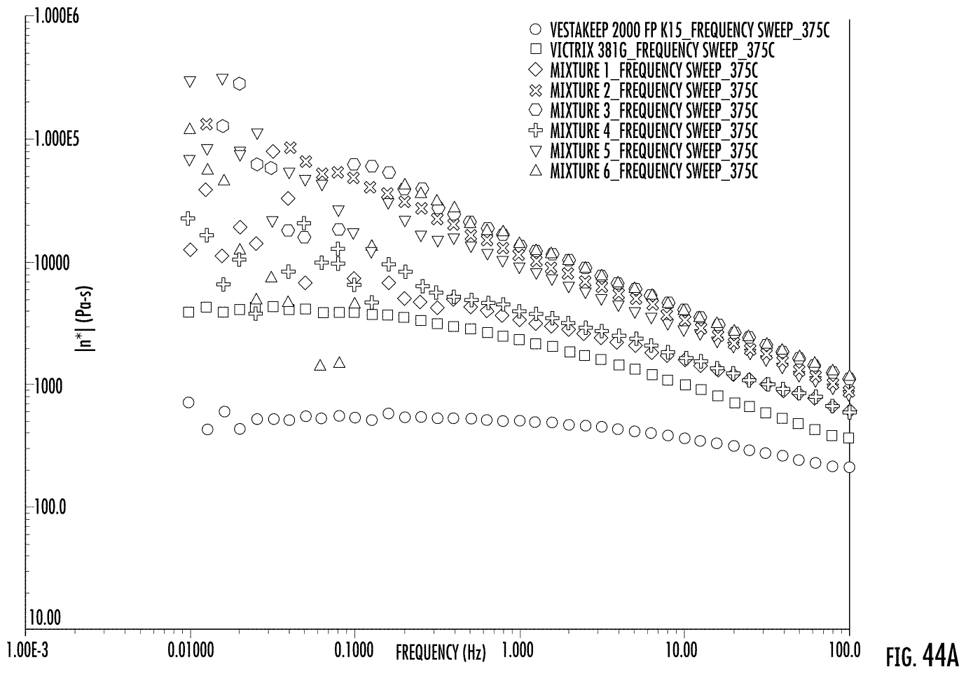

FIGS. 39-42 illustrate bioactivity at the surface of a device manufactured with 20% borate and 80% PEEK at seven days. FIGS. 39A and 39B illustrated the non-sanded device at seven days, and FIGS. 40A and 40B illustrate the sanded device at seven days. FIGS. 41A and 41B illustrate the non-sanded device at thirty-four days, and FIGS. 42A and 42B illustrate the sanded device at thirty-four days.

These figures confirm that 20% loading of either borate or 45S5 bioactive material with PEEK is sufficient for inducing hydroxyapatite formation on the composite's surface after seven and thirty-four days of bioactivity testing. In particular, they confirm that secondary processing of the device, such as sanding or otherwise machining the outer surface, induces hydroxyapatite formation around substantially the entire surface of the device within thirty-four days.

### Example 4

A composite material, or an implantable device made from a composite material, may be engineered from any suitable polymer for use in an implantable device, including but not limited to, a polyalkenoate, polycarbonate, polyamide, polyether sulfone (PES), polyphenylene sulfide (PPS), or a polyaryletherketone (PAEK), such as polyetheretherketone (PEEK) or polyetherketoneketone (PEKK). In other embodiments, the polymer may comprise a bioresorbable material, such as polyglycolic acid (PGA), poly-l-lactic acid (PLLA), poly-d-lactic acid, polycyanoacrylates, polyanhydrides, polypropylene fumarate and the like. The bioresorbable material may comprise all or only a portion of the polymer component and may, for example, be mixed or combined with a non-resorbable polymer.

In an exemplary embodiment, the polymer includes a composite of polyetheretherketone (PEEK) or polyetherketoneketone (PEKK) with particle sizes of about 0.5 to about 4,000 microns. The average diameter may be less than 1,000 microns. In other embodiments, the average diameter of the PAEK polymer is greater than 400 microns. In certain embodiments, the average diameter of the PAEK polymer is between 400 to 1,000 microns.

The bioactive additive may comprise any suitable bioactive material discussed above, such as borate glass powders and/or 45S5 glass powders from Mo-Sci Corporation of Rolla, Mo., each having particle sizes of between about 0.1 to about 2,000 microns. The average diameter of the bioactive glass and/or the boron-based material is between about 0.1 and about 400 microns, or about 50 to about 200 microns. In exemplary embodiments, the particle sizes may be about 90 to about 355 microns or about 75 to about 125 microns. The bioactive additive may comprise 100% borate, 100% 44S5 or a mixture of both (i.e., 50/50 or some other percentage). The overall composition of the device is about 80% PEEK, and 20% bioactive additive (i.e., borate and/or glass powders).

The device in this example is manufactured by producing composite pellets or other shapes of the PEEK particles and the bioactive materials. These composite pellets/shapes are then compression molded into a desired shape. The resulting product may also be subjected to secondary processing consisting sanding or other machining to increase surface exposure of bioactive glass.

### Example 5

A composite material, or an implantable device made from a composite material, is engineered from any of the materials described above. In this example, the polymer and bioactive materials comprise powders that are compression molded (as discussed above) to produce composite pellets or other shapes. These composite pellets/shapes are then injection molded into a desired shape. The resulting product may be subjected to secondary processing consisting sanding or other machining to increase surface exposure of bioactive glass.

### Example 6

A composite material, or an implantable device made from a composite material, is engineered from any of the materials described above. In this example, PEEK powder or pellets and bioactive glass, components are loaded into a screw extruder (single, twin, etc.) to produce homogeneous composite pellets. These homogeneous composite pellets are then compression molded into a desired shape. The resulting product may be subjected to secondary processing consisting sanding or other machining to increase surface exposure of bioactive glass.

### Example 7

A composite material, or an implantable device made from a composite material, is engineered from any of the materials described above. In this example, PEEK powder or pellets and bioactive glass, components are loaded into a screw extruder (single, twin, etc.) to produce homogeneous composite pellets. These homogeneous composite pellets are then injection molded into a desired shape. The resulting product may be subjected to secondary processing consisting sanding or other machining to increase surface exposure of bioactive glass.

### Example 8

A composite material, or an implantable device made from a composite material, is engineered from any of the materials described above. In this example, PEEK powder or pellets and bioactive material components are compounded using a screw extruder (single, twin, etc.) to produce homogenous composite 3D printable filaments (e.g., about 1.75 mm, 2.85 mm or 3.00 mm diameters).

The main part of the extruder is a barrel containing a screw (also sometimes referred to as an “auger” or a “drill”), which is connected to a heater (or heat chamber or heat element) towards its far end. On the other end, the screw is connected to an electric motor which will, via mechanical action, transport the resin pellets through the barrel towards the heater. Pellets are gravity-fed continuously from a hopper or similar feeding funnel. As the motor is continuously driving the auger, the resin pellets are pushed into the heater. The thermoplastic pellets will soften and melt because of the heat and are then pushed mechanically through a die. Pushing the soft thermoplastics through the die will cause it to form a continuous filament strand.

This homogeneous composite filament is then 3D printed using fused deposition modeling (FDM) into a desired product. In this method, a 3D model of the desired device is created using 3D modelling software, such Solidworks, Autodesk, PTC Creo and the like. The 3D model is then transformed into STL (standard tessellation language). STL files describe only the surface geometry of a three-dimensional object without any representation of color, texture or other common CAD model attributes. The STL file is then sliced into a .gcode file using slicing software, such as Cura, Simplify3D and the like. G-code is a commonly used computer numerical control programming language. G-code is mainly used in computer-aided additive manufacturing to automatically control manufacturing equipment. With 3D printing, g-code contains commands to move parts within the printer. The .gcode file is then sent to the 3D Printer for production.

The FDM 3D printer may include multiple print heads. Each print head is loaded with its own material that may contain a different percentage of bioactive material. In one example, the FDM 3D printer includes two print heads, with one print head containing a 40% by weight bioactive glass loaded filament and the other print head containing a 20% by weight bioactive glass loaded filament. These two filaments are printed together to product a composite object. Of course, it will be recognized that other configurations are possible. For example, the 3D printer may have three print heads, four print heads, or more. Each of the print heads may have the same of a different concentration of bioactive material therein.

FIG. 43 illustrates an example of a composite material or implantable device 600 manufactured according to these principles. As shown, device 600 includes an inner core 602 of material that includes 20% bioactive materials and 80% polymer. Surrounding the core 604 is a material that includes about 40% bioactive materials and 60% polymer. Surrounding inner core 602 is an outer portion 604 that contains 40% bioactive materials and 60% polymer. The outer portion 604 may be substantially annular such that the overall device is cylindrical. This device 600 was manufactured by extruding powder or pellets of bioactive material through a screw extruder (single, twin, etc.) to produce homogenous composite 3d printing filaments. The filaments were then 3D printed with two separate print heads such that inner core 602 contains 20% bioactive materials and outer portion 604 contains 40% bioactive materials.

The resulting product may be subjected to secondary processing consisting annealing, sanding or machining to increase surface exposure of bioactive glass.

### Example 9

A composite material, or an implantable device made from a composite material, is engineered from any of the materials described above. In this example, powders of polymer and bioactive material are compression molded to produce composite pellets or other shapes. These composite pellets are then compounded using a screw extruder (single, twin, etc.) to produce homogenous composite pellets. These homogeneous composite pellets are then compression molded into a desired shape. The resulting product may be subjected to secondary processing consisting sanding or other machining to increase surface exposure of bioactive glass.

### Example 10

A composite material, or an implantable device made from a composite material, is engineered from any of the materials described above. In this example, powders of polymer and bioactive material are compression molded of to produce composite pellets or other shapes. These composite pellets are then compounded using a screw extruder (single, twin, etc.) to produce homogenous composite pellets. These homogeneous composite pellets are then injection molded into a desired shape. The resulting product may be subjected to secondary processing consisting sanding or other machining to increase surface exposure of bioactive glass.

### Example 11

A composite material, or an implantable device made from a composite material, is engineered from any of the materials described above. In this example, powders of polymer and bioactive material are compression molded of to produce composite pellets or other shapes. These composite pellets are then compounded using a screw extruder (single, twin, etc.) to produce homogenous composite 3d printing filament (e.g., about 1.75- and 2.85-mm diameters). This homogeneous composite filament is then 3D printed using fused deposition modeling (FDM) into a desired product. The resulting product may be subjected to secondary processing consisting sanding or other machining to increase surface exposure of bioactive glass.

### Example 12

A composite material, or an implantable device made from a composite material, is engineered from any of the materials described above. In this example, polymer and bioactive glass powders are premixed. The mixed powder is then dispersed in a thin layer on top of a platform inside of the build chamber. The printer preheats the powder to a temperature just below the melting point of the raw material. This makes it easier for the laser beam to raise the temperature of specific regions of the powder bed as it traces the model to solidify a part. The laser scans a cross-section of the 3D model, heating the powder to just below or right at the melting point of the material. This fuses the particles together mechanically to create one solid part. The unfused powder supports the part during printing and eliminates the need for dedicated support structures.

The build platform lowers by one layer into the build chamber, typically between 50 to 200 microns, and a recoater applies a new layer of powder material on top. The laser then scans the next cross-section of the build. This process repeats for each layer until parts are complete, and the finished parts are left to cool down gradually inside the printer. Once the parts have cooled, the operator removes the build chamber from the printer and transfers it to a cleaning station, separating the printed parts and cleaning of the excess powder.

### Example 13

A composite material, or an implantable device made from a composite material, is engineered from any of the materials described above. In this example, particles of a polymer and bioactive materials are separated loaded into two different extruders. The particles are co-extruded to form a composite material.

### Example 14

A composite material, or an implantable device made from a composite material, is engineered from any of the materials described above. In this example, particles of PEEK and borate bioactive glass were compounded using a twin screw extruder. In two separate examples, the borate bioactive glass was included at 25% and 30% by weight, respectively. The extruder was operating at 125 RPM and outputted a composite filament comprising borate and PEEK. The temperature of the system ranged from about 260 degrees Celsius to about 400 degrees Celsius during the process. In addition to the extruder, two sidescrews were used for the twin screw extrusion process. The sidescrews operated between 0 and 200 RPM.

### Example 15

In other process according to the present disclosure, various formulations of bioactive composites that includes mixtures of multiple materials (e.g., x, y, z, etc.) are verified for later processing by mapping the viscosity profiles of the mixtures through rheology testing. A rheometer is a laboratory device used to measure the way in which a liquid, suspension or slurry flows in response to applied forces. It is used for those fluids which cannot be defined by a single value of viscosity and therefore require more parameters to be set and measured than is the case for a viscometer. It measures the rheology of the fluid.

Rheology testing was performed on various PEEK and bioactive material compositions in order to understand how one composition's viscosity compares to others. After understanding the composition's viscosity, select compositions were manufactured into cylinders/pellets using powder compression molding, as discussed above. Bioactivity testing is then performed on the produced pellets. If bioactivity testing passes, the composition is moved to twin screw extrusion (discussed above). If bioactivity fails, the composition is reworked and rheology testing is performed again on a new composition. This loop is repeated until the composition passes bioactivity.

To verify that a polymer, such as Vestakeep 2000 FP K15 (polyether ether ketone fine powder), is processable in a twin screw extruder after being mixed with bioactive materials, a TA AR2000ex parallel plate rheometer with a diameter of 25 mm was used to characterize the rheological performance of pure PEEK powder and mixtures of PEEK powder and bioactive powders. The test specimens included compression molded discs with a diameter of 1.5 inches and thickness of 0.14 inches. For reference, Victrex 381G (3D printable grade of PEEK) and Vestakeep 2000 FP K15 were tested as controls. The bioactive powders used were MoSci borate powders and/or MoSci 45S5 glass powders. Each of the borate and 45S5 had two different versions: larger diameter powders having a diameter of about 90 to about 355 microns and smaller diameter powders with a diameter of about 75 to about 125 microns.

Table 1 illustrates the compositions of each mixture tested.

| | | Vestakeep | Large | Large | Small | Small |
|---|---|---|---|---|---|---|
| | Victrex | 2000 FP | 45S5 | Borate | 45S5 | Borate |
| | 381G | K15 | Powders | Powders | Powders | Powders |
| Control | 100% | 0% | 0% | 0% | 0% | 0% |
| Pure PEEK | 0% | 100% | 0% | 0% | 0% | 0% |
| Mixture 1 | 0% | 80% | 20% | 0% | 0% | 0% |
| Mixture 2 | 0% | 75% | 25% | 0% | 0% | 0% |
| Mixture 3 | 0% | 70% | 30% | 0% | 0% | 0% |
| Mixture 4 | 0% | 80% | 0% | 20% | 0% | 0% |
| Mixture 5 | 0% | 75% | 0% | 25% | 0% | 0% |
| Mixture 6 | 0% | 70% | 0% | 30% | 0% | 0% |
| Mixture 7 | 0% | 75% | 0% | 0% | 25% | 0% |
| Mixture 8 | 0% | 70% | 0% | 0% | 30% | 0% |
| Mixture 9 | 0% | 75% | 0% | 0% | 0% | 35% |
| Mixture 10 | 0% | 70% | 0% | 0% | 0% | 30% |

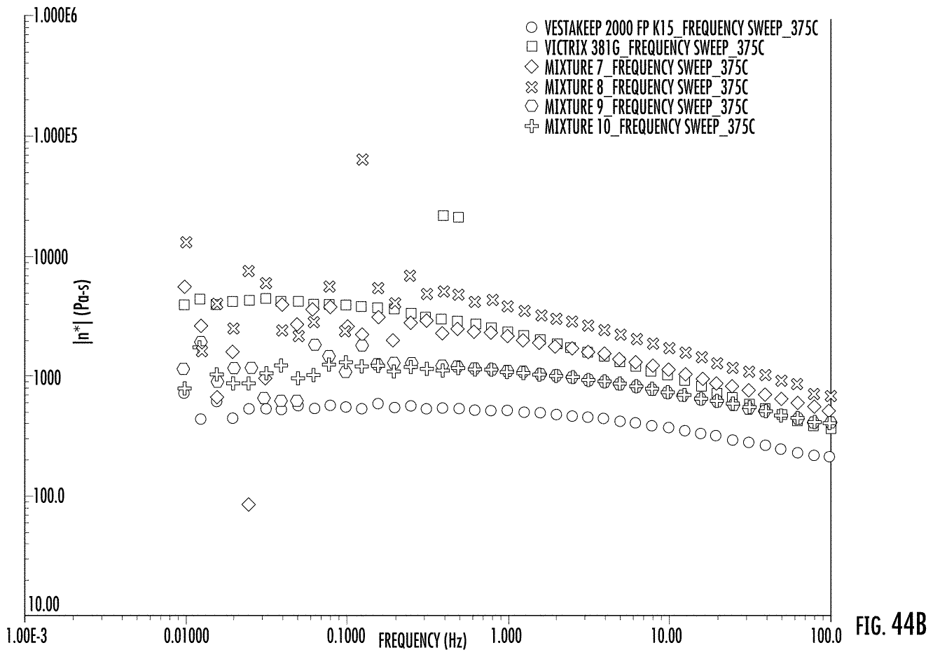

FIG. 44A illustrates the viscosities over time for the larger powder mixtures with the Victrex 381G PEEK and the Vestakeep 200 FP K15 PEEK as controls. Pure Vestakeep 2000 FP K15 has the lowest viscosity. Adding larger powders of bioactive material increases the viscosities of the mixtures. At the same loading level, the mixtures of larger powder 45S5 with PEEK and larger powder borate with PEEK have substantially the same viscosities. The mixtures with three different loading levels (20%, 25% and 30% bioactive materials by weight) of larger glass powders all have higher viscosities than Victrex 381G. The mixtures at 20% bioactive materials by weight have slightly higher viscosities than Victrex 381G, but are still processable in the twin screw extruder at 375° C. The mixtures with 25% and 30% bioactive materials by weight have viscosities that may not be easily processable in the twin screw extruder at 375° C. Accordingly, it has been found that lower viscosity PEEK material, such as Vestakeep 1000, should be used if the loading level of larger glass powders are above 20% bioactive material by weight, e.g., 25% or 30%.

FIG. 44B illustrates the viscosities over time for the smaller diameter powder mixtures and the Victrex 381G PEEK and the Vestakeep 2000 FP K15 PEEK as controls. As shown, the mixture with 25% 45S5 glass powders by weight has a similar viscosity as Victrex 381G. The mixtures with 30% 45S5 glass powders by weight have a slightly higher viscosity than Victrex 381G. The mixtures with 25% and 30% borate powders by weight have substantially the same or lower viscosities than Victrex 381G. Accordingly, it has been found that all of the mixtures of small glass powder tested are processable with Victrex 381G PEEK in a twin screw extruder at 375° C.

Other embodiments of the invention will be apparent to those skilled in the art from consideration of the specification and practice of the disclosure provided herein. It is intended that the specification and examples be considered as exemplary only, with a true scope and spirit of the disclosure being indicated by the following claims.

1-37. (canceled)

38. An implantable device, comprising:
a main body; and
a bioactive component comprising a polyarylretherketone (PAEK) polymer component and a bioactive additive component incorporated substantially throughout the polymer component.

39. The implantable device of claim 38, wherein the bioactive additive component comprises a silica-based bioactive glass, a boron-based bioactive material, a strontium-based bioactive material or a combination thereof.

40. The implantable device of claim 39, wherein the boron-based bioactive material comprises borate particles.

41. The implantable device of claim 39, wherein the silica-based glass additive comprises 45S5 bioactive materials or Combeite.

42. The implantable device of claim 38, wherein the main body is formed from a framework, and the bioactive component is incorporated on, or into, at least a portion of the framework.

43. The implantable device of claim 38, wherein the main body comprises polyetheretherketone (PEEK) or polyetherketoneketone (PEKK).

44. The implantable device of claim 38, wherein the main body comprises a polymer, a metal or a ceramic material.

45. The implantable device of claim 38, wherein the main body comprises an outer surface and further comprising a second bioactive component disposed on, or around, said outer surface.

46. The implantable device of claim 38, wherein the main body comprises one or more internal spaces and the bioactive component is disposed adjacent to, or within, the internal spaces.

47. The implantable device of claim 38, wherein the main body comprises one or more internal surfaces and the bioactive component is disposed on, or adjacent to, the internal surfaces.

48. The implantable device of claim 38, wherein the bioactive component comprises one or more bundles of particles disposed within, or on, the main body.

49. The implantable device of claim 38, wherein the bioactive component is incorporated throughout the main body.

50. The implantable device of claim 38, wherein the main body and the bioactive additive component are formed from particles mixed together into a substantially homogenous composite.

51. The implantable device of claim 38, further being porous.

52. The implantable device of claim 38, further being non-porous.

53. The implantable device of claim 38, wherein the main body is formed from a lattice structure.

54. The implantable device of claim 38, further comprising one or more bioactive elements comprising a polymer and a bioactive additive material, the bioactive elements extending through at least a portion of the main body.

55. The implantable device of claim 54, wherein the bioactive elements comprise a substantially cylindrical shape and extend from one end of the main body to an opposite end of the main body

56. The implantable device of claim 55, wherein the bioactive elements extend in a substantially parallel direction with each other.

57. The implantable device of claim 38, wherein the bioactive component comprises fibers and the main body comprises pores.

58. The implantable device of claim 57, wherein the pores extend in a direction substantially parallel to the fibers.

59. The implantable device of claim 57, wherein the pores extend along a length of the fibers.

60. The implantable device of claim 57, wherein the main body has a first surface and a second surface opposite the first surface, wherein the pores extend from the first surface to the second surface.

61. The implantable device of claim 57, wherein the fibers form one or more rods extending from the first surface to the second surface.

62. The implantable device of claim 57, wherein the fibers comprise a material configured to promote the circulation of liquids between the fibers, and wherein the fibers are configured to promote capillary action between aligned fibers to pull fluids therethrough.

63. The implantable device of claim 38, further being an orthopedic implant, a spinal fusion implant, dental implant, total or partial joint replacement or repair device, trauma repair device, bone fracture repair device, reconstructive surgical device, alveolar ridge reconstruction device, or veterinary implant.

64. The implantable device of claim 38, wherein the bioactive component comprises an outer surface and an interior, wherein the outer surface includes a higher concentration of bioactive material additive than the interior.

65. The implantable device of claim 64, wherein the outer surface comprises about 40 to about 100 percent bioactive material additive and about 0 to about 60 percent polymer and the interior comprises about 5 to about 40 percent bioactive material additive and about 60 to about 95 percent polymer.

66. The implantable device of claim 64, wherein the outer surface comprises about 75 to about 100 percent bioactive material additive and about 0 to about 25 percent polymer and the interior comprises about 5 to about 25 percent bioactive material additive and about 75 to about 95 percent polymer.

67-120. (canceled)
